(19) United States
(12) Patent Application Publication
(10) Pub. No.: US 2024/0264155 A1
(43) Pub. Date: Aug. 8, 2024

(54) METHODS AND COMPOSITIONS FOR IN SITU ANALYSIS USING DETECTABLY LABELED MAGNETIC PARTICLES

(71) Applicant: 10x Genomics, Inc., Pleasanton, CA (US)

(72) Inventor: Justin COSTA, Union City, CA (US)

(21) Appl. No.: 18/429,226

(22) Filed: Jan. 31, 2024

Related U.S. Application Data
(60) Provisional application No. 63/482,729, filed on Feb. 1, 2023.

(51) Int. Cl. G01N 33/543; C12Q 1/6844

(52) U.S. Cl. CPC G01N 33/54333; C12Q 1/6844; C12Q 2600/166

(57) ABSTRACT

The present disclosure relates in some aspects to methods and compositions for analyzing a biological sample. In some aspects, provided herein are detectably labeled magnetic particles, methods, and kits for analyzing a biological sample (e.g., for detecting a target nucleic acid at a location in the biological sample).

### CROSS-REFERENCE TO RELATED APPLICATIONS

This application claims priority to U.S. Provisional Patent Application No. 63/482,729, filed Feb. 1, 2023, entitled “Methods and Compositions for in situ Analysis using Detectably Labeled Magnetic Particles,” which is herein incorporated by reference in its entirety for all purposes.

### FIELD

The present disclosure relates in some aspects to methods and compositions for in situ analysis of an analyte in a biological sample using detectably labeled magnetic particles.

### BACKGROUND

Single molecule fluorescent in situ hybridization (smFISH), including amplified smFISH methods such as hybridization chain reaction (HCR), are used to determine expression levels of analytes, such as RNA. One limitation of these approaches is that the signals may be dim and diffuse. Enhancement of signal brightness has largely relied on colocalization of numerous molecular signals on a target, which can result in signal diffusion. Furthermore, removal of an initial probe through chemical or enzymatic means in order to apply a second probe can be laborious and time-consuming. Thus, improved methods are needed. The present disclosure addresses this and other needs.

### SUMMARY

In some aspects, provided herein is a method for analyzing a biological sample, comprising: a) contacting the biological sample with a detectably labeled magnetic particle, wherein the detectably labeled magnetic particle comprises: (i) a magnetic core, (ii) a detectable label, and (iii) oligonucleotides comprising a reporter hybridization sequence, wherein the reporter hybridization sequence is complementary to a reporter sequence present in a plurality of intermediate probes bound directly or indirectly to a target nucleic acid molecule in the biological sample, whereby the magnetic particle hybridizes to the reporter sequence via one or more of the oligonucleotide molecules and is thereby associated with the target nucleic acid molecule; and b) detecting the detectably labeled magnetic particle at a location in the biological sample. The detectably labeled magnetic particle can be manufactured synthetically or biologically in a cost-effective manner. In some embodiments, ^{CNV}K oligos can be used to photo-crosslink the detectably labeled magnetic particle to intermediate probes for simultaneous removal of the magnetic particle and intermediate probes within a time frame on the order of seconds. Further, this method overcomes the current limitations of having dim and diffuse signal in nucleic acid detection, by leveraging proximity enforced hybridization of nucleic acids, e.g., rolling circle amplification products, thereby compacting the signal. In some embodiments, the magnetic particles can be leveraged for multiplex detection, using a universal pool of detectably labeled magnetic particles with catalogued pairs of fluorescence signals and reporter hybridization sequences. In some embodiments, a plurality of moieties for different functionalities are attached to the magnetic particle via encoded coupling chemistry (e.g., for detection and removal).

In some aspects, provided herein is a method for analyzing a biological sample, comprising: a) contacting the biological sample with a detectably labeled magnetic particle, wherein the detectably labeled magnetic particle comprises: (i) a magnetic core, (ii) a detectable label, and (iii) oligonucleotides comprising a reporter hybridization sequence, wherein the reporter hybridization sequence is complementary to a reporter sequence present in a plurality of intermediate probes bound directly or indirectly to a target nucleic acid molecule in the biological sample, whereby the magnetic particle hybridizes to the reporter sequence of one or more of the intermediate probes via one or more of the oligonucleotide molecules and is thereby associated with the target nucleic acid molecule; and b) detecting the detectably labeled magnetic particle at a location in the biological sample.

In some of any of the embodiments herein, the detectably labeled magnetic particle comprises a coating or lipid membrane comprising a plurality of coupling agent molecules, wherein at least a subset of the coupling agent molecules are coupled with a binding partner associated with the oligonucleotide molecules. In some of any of the embodiments herein, at least a subset of the coupling agent molecules is coupled with a binding partner associated with the detectable label. In some of any of the embodiments herein, the coupling agent is a biotinylated lipid. In some of any of the embodiments herein, the coupling agent comprises a biotin-modified protein in the coating or lipid membrane. In some of any of the embodiments herein, the binding partner associated with the oligonucleotide molecules comprise avidin, streptavidin, or neutravidin. In some embodiments, the oligonucleotide molecules are associated with the binding partner via biotin. In some of any of the embodiments herein, the binding partner associated with the detectable label comprises avidin, streptavidin, or neutravidin. In any of the embodiments herein, the detectable label can be associated with the binding partner via biotin.

In any of the embodiments herein, the coupling agent can be or can be fused to a protein in the protein coating or lipid membrane. In some of any of the embodiments herein, the coupling agent is selected from the group consisting of avidin, streptavidin, and neutravidin. In some of any of the embodiments herein, the binding partner associated with the oligonucleotide molecules comprises biotin. In some of any of the embodiments herein, the binding partner associated with the detectable label comprises biotin.

In some of any of the embodiments herein, the detectable label comprises a fluorescent protein. In any of the embodiments herein wherein the magnetic particle comprises a lipid membrane, and the fluorescent protein can be fused to a protein in the lipid membrane. In some of any of the embodiments herein, the detectable label comprises a fluorophore. In any of the embodiments herein, at least a subset of the oligonucleotide molecules can be detectably labeled. In some of any of the embodiments herein, the detectably labeled magnetic particle comprises at least 50, at least 100, at least 200, or at least 300 fluorophores. In some of any of the embodiments herein, at least 100, at least 500, or least 1000 oligonucleotides are coupled to the coupling agent molecules on the detectably labeled magnetic particle.

In some of any of the embodiments herein, the method comprises: c) removing the detectably labeled magnetic particle and the plurality of intermediate probes from the biological sample after detecting the detectably labeled magnetic particle e.g., in b). In some of any of the embodiments herein, removing the detectably labeled magnetic particle comprises applying a magnetic field to the biological sample. In some of any of the embodiments herein, removing the detectably labeled magnetic particle comprises performing a wash (e.g., a stringent wash).

In some of any of the embodiments herein, the oligonucleotide molecules individually are functionalized with a crosslinkable moiety. In some of any of the embodiments herein, the method comprises crosslinking the reporter hybridization sequences to the hybridized one or more intermediate probes before removing the detectably labeled magnetic particle. In any of the embodiments herein, the crosslinkable moiety can be a modified nucleoside in the reporter hybridization sequence or is connected to a nucleotide residue in the reporter hybridization sequence. In any of the embodiments herein, crosslinking can occur between the reporter hybridization sequence and the reporter sequence of the hybridized intermediate probe. In any of the embodiments herein, the crosslinkable moiety can be configured to crosslink to a nucleobase of the hybridized intermediate probe. In some of any of the embodiments herein, the method comprises irradiating the biological sample to photo-activate the crosslinkable moiety. In some of any of the embodiments herein, the biological sample is irradiated using a 350-400 nm wavelength of light. In some of any of the embodiments herein, the nucleobase is a thymine, uridine, or cytosine. In some of any of the embodiments herein, the nucleobase is an adenine.

In some of any of the embodiments herein, crosslinkable moiety is connected to the nucleotide residue via a linker. In some of any of the embodiments herein, the crosslinkable moiety comprises a vinylcarbazone-based moiety. In any of the embodiments herein, the crosslinkable moiety can be a 3-cyanovinylcarbazole (^{CNV}K) nucleoside, a 3-cyanovinylcarbazole modified D-threoninol (^{CNV}D), a pyranocarbazole nucleoside (^{PC}X) or a pyranocarbazole modified D-threoninol (^{PCX}D). In any of the embodiments herein, the crosslinkable moiety can be a 3-cyanovinylcarbazole phosphoramidite or a pyranocarbazole phosphoramidite. In any of the embodiments herein, the crosslinkable moiety is a psoralen or a psoralen derivative. In some embodiments, the psoralen is a C2 psoralen. In any of the embodiments herein, the crosslinkable moiety can be a psoralen C2 phosphoramidite. In any of the embodiments herein, the crosslinkable moiety can be a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite). In any of the embodiments herein, the crosslinkable moiety can be a 5′-Dimethoxytrityl-5-iodo-2′-deoxyUridine,3′-[(2-cyanoethyl)-(N,N-diisopropyl)]-phosphoramidite (5-I-dU-CE phosphoramidite). In some of any of the embodiments herein, the oligonucleotide molecules individually comprise two or more nucleotide residues functionalized with crosslinkable moieties in the hybridization region.

In some of any of the embodiments herein, the method comprises: d) binding a second plurality of intermediate probes comprising a second reporter sequence directly or indirectly to the target nucleic acid molecule, e) contacting the biological sample with a second detectably labeled magnetic particle, wherein the second detectably labeled magnetic particle comprises: (i) a magnetic core, and (ii) a coating or lipid membrane comprising a plurality of coupling agent molecules, wherein at least a subset of the coupling agent molecules are coupled with oligonucleotides comprising a second reporter hybridization sequence, wherein the second reporter hybridization sequence is complementary to the second reporter sequence, whereby the magnetic particle binds to the second reporter sequence via one or more of the oligonucleotide molecules and is thereby associated with the target nucleic acid molecule; and f) detecting the second detectably labeled magnetic particle at the location in the biological sample.

In any of the embodiments herein, oligonucleotide molecules of the detectably labeled magnetic particle can hybridize to multiple intermediate probes of the plurality of intermediate probes comprising the reporter sequence. In some of any of the embodiments herein, a single detectably labeled magnetic particle is associated with the target nucleic acid molecule. In some of any of the embodiments herein, the method comprises detecting the single detectably labeled magnetic particle associated with the target nucleic acid molecule at the location in the biological sample. In any of the embodiments herein, two or more detectably labeled magnetic particles can be associated with the target nucleic acid molecule at the location in the biological sample. In some of any of the embodiments herein, the method comprises detecting the two or more detectably labeled magnetic particles associated with the target nucleic acid molecule at the location in the biological sample.

In some of any of the embodiments herein, the magnetic core comprises a paramagnetic material. In some of any of the embodiments herein, the magnetic core comprises a ferromagnetic material. In some embodiments, the ferromagnetic material is selected from the group consisting of iron, cobalt, nickel, alloys thereof, and combinations thereof. In some of any of the embodiments herein, the magnetic core comprises iron oxide. In some of any of the embodiments herein, the magnetic core comprises Fe²⁺Fe³⁺₂O₄. In some of any of the embodiments herein, the magnetic core comprises a magnetite crystal. In some of any of the embodiments herein, the magnetic core is produced by plasma spark ablation of iron oxides.

In some of any of the embodiments herein, the detectably labeled magnetic particle comprises a modified magnetosome derived from magnetotactic bacteria. In some of any of the embodiments herein, the coupling agent is operably linked to a magnetosome membrane protein selected from the group consisting of: mamG, mamF, mamD, mamA, and mamC. In any of the embodiments herein, the magnetosome membrane protein can be mamC protein. In any of the embodiments herein, the magnetotactic bacteria can be engineered to express the magnetosome membrane protein fused to the capture agent.

In some of any of the embodiments herein, the target nucleic acid molecule is a rolling circle amplification (RCA) product. In some of any of the embodiments herein, the RCA product is generated from a circular probe or circularizable probe or probe set bound directly or indirectly to a nucleic acid analyte, product, or labeling agent in the biological sample. In any of the embodiments herein, the nucleic acid analyte can be RNA. In some embodiments, the nucleic acid analyte is mRNA. In any of the embodiments herein, the labeling agent can be bound to a non-nucleic acid analyte in the biological sample. In some of any of the embodiments herein, the rolling circle amplification product comprises multiple copies of a barcode sequence. In some of any of the embodiments herein, the plurality of intermediate probes comprises a barcode recognition sequence complementary to the barcode sequence.

In some of any of the embodiments herein, the target nucleic acid molecule is a nucleic acid analyte or a product thereof. In any of the embodiments herein, the target nucleic acid molecule can be RNA. In some embodiments, the target nucleic acid molecule is mRNA. In any of the embodiments herein, the target nucleic acid molecule can be cDNA. In any of the embodiments herein, the plurality of intermediate probes can bind directly or indirectly to a plurality of target sequences in the target nucleic acid molecule. In some of any of the embodiments herein, the plurality of intermediate probes comprises a plurality of target recognition sequences complementary to a plurality of target sequences in the target nucleic acid molecule.

In some of any of the embodiments herein, detecting the detectably labeled magnetic particle at a location in the biological sample comprises detecting a single detectably labeled magnetic particle hybridized to the plurality of intermediate probes bound directly or indirectly to the target nucleic acid molecule. In some of any of the embodiments herein, the magnetic core of the detectably labeled magnetic particle has a diameter of between about 5-10 nm, 10-20 nm, 20-50 nm, or 50-150 nm.

In some of any of the embodiments herein, contacting the biological sample with the detectably labeled magnetic particle comprises contacting the biological sample with a pool of detectably labeled magnetic particles comprising different detectable labels. In any of the embodiments herein, the detectably labeled magnetic particles may have less than 10% variation in diameters. In any of the embodiments herein, the magnetic cores of the detectably labeled magnetic particles may have an average diameter of between about 10 nm and about 50 nm. In any of the embodiments herein, the magnetic cores of the detectably labeled magnetic particles may have an average diameter of between about 50 nm and about 100 nm.

In some aspects, provided herein is a method for analyzing a biological sample comprising a plurality of target nucleic acid molecules, the method comprising: a) contacting the sample with a panel of first intermediate probes and a pool comprising multiple species of detectably labeled magnetic particles, wherein each species of detectably labeled magnetic particles comprises: (i) a magnetic core, (ii) a different detectable label, and (iii) oligonucleotides comprising a reporter hybridization sequence corresponding to the detectable label; wherein each of a plurality of the first intermediate probes comprises (i) a recognition sequence complementary to a target sequence in a target nucleic acid molecule of the plurality of target nucleic acid molecules and (ii) a reporter sequence complementary to one of the reporter hybridization sequences in the pool of detectably labeled magnetic particles, b) imaging the sample to detect a first signal from a complex formed between a detectably labeled magnetic particle of the pool of detectably labeled magnetic particles, a first intermediate probe of the panel of first intermediate probes, and a target nucleic acid of the plurality of target nucleic acid molecules; c) removing the first intermediate probe and the detectably labeled magnetic particle from the target nucleic acid molecule; d) contacting the sample with a panel of second intermediate probes and the pool of detectably labeled magnetic particles, wherein each of a plurality of the second intermediate probes comprises (i) a recognition sequence complementary to a target sequence in a target nucleic acid molecule of the plurality of target nucleic acid molecules and (ii) a reporter sequence complementary to one of the reporter hybridization sequences in the pool of detectably labeled magnetic particles; and e) imaging the sample to detect a second signal from a complex formed between a detectably labeled magnetic particle of the pool of detectably labeled magnetic particles, a second intermediate probe of the panel of second intermediate probes, and the target nucleic acid.

In any of the embodiments herein, the first signal and the second signal can be part of a sequence of signals that identifies the target nucleic acid molecule. In any of the embodiments herein, the plurality of target nucleic acid molecules can be a plurality of rolling circle amplification products produced in the biological sample. In any of the embodiments herein, the rolling circle amplification products can be associated with nucleic acid or non-nucleic acid analytes in the biological sample. In some of any of the embodiments herein, removing the first intermediate probe and the detectably labeled magnetic particle from the target nucleic acid molecule comprises applying a magnetic field to the biological sample. In any of the embodiments herein, the detectable labels can be fluorescent proteins or fluorophores. In any of the embodiments herein, the sequence of signals can be a sequence of fluorescent signals.

In any of the embodiments herein, the biological sample can be non-homogenized. In any of the embodiments herein, the biological sample can be selected from the group consisting of a formalin-fixed, paraffin-embedded (FFPE) sample, a frozen tissue sample, and a fresh tissue sample. In any of the embodiments herein, the biological sample can be permeabilized. In any of the embodiments herein, the biological sample can be embedded in a matrix. In some of any of the embodiments herein, the matrix comprises a hydrogel. In any of the embodiments herein, the biological sample can be cleared. In any of the embodiments herein, the biological sample can be a tissue slice between about 1 μm and about 50 μm in thickness. In any of the embodiments herein, the biological sample can be a tissue slice between about 5 μm and about 35 μm in thickness. In any of the embodiments herein, the biological sample is on a substrate. In some embodiments, the substrate is an optically transparent planar substrate, such as a glass slide or coverslip.

In some aspects, provided herein is a magnetic particle, comprising: (i) a magnetic core, (ii) a detectable label, and (iii) a plurality of oligonucleotides comprising a reporter hybridization sequence, wherein the reporter hybridization sequence corresponds to the detectable label.

In some aspects, provided herein is a kit, comprising: a) at least four different species of detectably labeled magnetic particles, wherein each species of detectably labeled magnetic particles comprises: (i) a magnetic core, (ii) a different detectable label, and (iii) oligonucleotides comprising a reporter hybridization sequence corresponding to the detectable label; and b) a sequential series of intermediate probe panels, wherein each panel comprises multiple intermediate probe species, and wherein each intermediate probe species comprises (i) a barcode recognition sequence complementary to a different barcode sequence, and (ii) a reporter sequence, which can be the same or different from the reporter sequence of a different intermediate probe species in the panel; wherein the reporter sequence is complementary to the reporter hybridization sequence of one of the species of detectably labeled magnetic particles.

### BRIEF DESCRIPTION OF THE DRAWINGS

The drawings illustrate certain features and advantages of this disclosure. These embodiments are not intended to limit the scope of the appended claims in any manner.

FIG. 1 depicts an exemplary detectably labeled magnetic particle and an exemplary plurality of intermediate probes bound to a target nucleic acid molecule.

FIG. 2 illustrates an exemplary method of analyzing a biological sample. As illustrated, a detectably labeled magnetic particle can be associated with a target nucleic acid molecule via hybridization to one or more intermediate probes bound to the target nucleic acid molecule, and detected. The detectably labeled magnetic particle can then be removed, and the sample can be contacted with a second plurality of intermediate probes and a second detectably labeled magnetic particle.

FIG. 3 depicts an exemplary pool of different species of detectably labeled magnetic particles.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Methods of analyzing the locations and identities of analytes in a biological sample provide important information regarding cell types and functions in numerous contexts. Multiplexed analyte detection by sequential hybridization of detectably labeled probes allows simultaneous analysis of a large number of analytes in the same biological sample. However, improved methods are needed to provide improved signal intensity and improved signal-to-noise ratios. Additionally, the number of sequential cycles of hybridization that can be performed to identify a target nucleic acid sequence may be limited in some cases by the ability to efficiently remove hybridized probes from prior cycles, which can be time consuming and/or damaging to the biological sample.

In some aspects, provided herein are methods of analyzing a biological sample using detectably labeled magnetic particles. In some embodiments, the detectably labeled magnetic particles comprise at least 20, 50, 100, 200, or 500 or more detectable labels such as fluorophores, thus providing a very bright signal. In some embodiments, the magnetic core of the particle has a diameter of less than 100 nm, thus providing a compact signal. In some embodiments, a single detectably labeled magnetic particle is detected at a location in the biological sample. In some embodiments, the single detectably labeled magnetic particle comprises a plurality of oligonucleotide molecules hybridized to a plurality of intermediate probes bound to a target nucleic acid molecule in the biological sample. In some embodiments, hybridization of the multiple oligonucleotides to multiple intermediate probes stabilizes the detectably labeled magnetic particle's association with the target nucleic acid molecule.

In some embodiments, the hybridization of oligonucleotides on the detectably labeled magnetic particles to the intermediate probes bound to the target nucleic acid molecule is biased by proximity. Without being bound by theory, in some cases, the detectably labeled magnetic particles and methods disclosed herein have the advantage of proximity biased hybridization: in effect, oligonucleotides on the particle may hybridize to the nearest intermediate probe on the target nucleic acid molecule (e.g., a rolling circle amplification product), drawing in the larger molecular complex to promote more intermediate probe binding to the oligonucleotides on the detectably labeled magnetic particle in a cascade. In any of the embodiments herein, the effect may simultaneously compact the observable size of each target nucleic acid molecule (e.g., rolling circle amplification product), while giving discrete levels of fluorescence for each target nucleic acid molecule (in some embodiments, binding of one detectably labeled magnetic particle will be half as bright compared to two detectably labeled magnetic particles binding, etc.). Non-specifically bound detectably labeled magnetic particles can then be removed from the biological sample prior to detection (e.g., imaging).

In some embodiments, the detectably labeled magnetic particles and methods provided herein further have the advantage of being able to remove (e.g., strip) the detectably labeled magnetic particles with application of magnetic fields. In some embodiments, the oligonucleotides of the detectably labeled magnetic probes can comprise photoreactive crosslinkers. In some embodiments, the method comprises covalently crosslinking the oligonucleotides to the hybridized intermediate probes (e.g., during DAPI imaging). In some embodiments, covalently crosslinking the oligonucleotides to the hybridized intermediate probes allows efficient removal of the intermediate probes together with the detectably labeled magnetic particle.

In some aspects, provided herein are methods of making detectably labeled magnetic particles. In some embodiments, the detectably labeled magnetic particles are modified magnetosomes. In some aspects, the provided methods of manufacturing detectably labeled magnetosomes provide particles of relatively uniform shape and size that can be cheaply and easily manufactured at scale. In some embodiments, the resulting particles are easily dispersed in water and have facile programmable surface chemistry. In some embodiments, the detectably labeled magnetic particles possess stable single domain magnetic moments at physiological temperature. In some embodiments, the methods of making detectably labeled magnetic particles provided herein provide a high degree of flexibility for attachment of different detectable labels, oligonucleotides, and combinations thereof.

In some aspects, provided herein is a method of producing a detectably labeled magnetic particle from a modified magnetotactic microorganism (e.g., magnetotactic bacteria). In some embodiments, the magnetotactic microorganisms biomineralize magnetite (Fe²⁺Fe³⁺₂O₄.). In some embodiments, the magnetite, an oxide of iron that contains both divalent ferrous and trivalent ferric species in the lattice. Species that synthesize magnetosomes tend to inhabit microoxic environments. In some embodiments, the magnetosomes are arranged in chains. In some embodiments, the magnetosomes do not arrange in chains. In some instances, the magnetosome structures endow cellular dipole moments large enough that their interaction with Earth's magnetic field overcomes the randomizing thermal forces that orient and disperse other aquatic microbes. In some embodiments, the magnetosome comprises a magnetic core enveloped by a lipid membrane. In some embodiments, the present disclosure leverages the lipid membrane for its advantages for purification and stability. Due to the abundance of phosphatidylserine in the membrane, magnetosomes have a net negative charge and are easily dispersed in aqueous environments.

In some embodiments, the methods of making detectably labeled magnetic particles provided herein harness the proteomics of the lipid envelope of the magnetosome for production of modified magnetosomes. Depending on the species of magnetotactic bacteria, twenty to thirty magnetosome-membrane specific proteins may be used by the cell to manufacture the magnetosome. These genes are typically arranged across three operons and encode proteins responsible for the control of vesicle formation, ion transport (including large iron species), and the biomineralization of perfectly stochiometric magnetite crystals. The most well studied magnetosome proteins are encoded by the Mam family of genes. The MamC protein is one of the most abundant in the magnetosome membrane, but several others in this family have also been shown to be successful at biomineralization while fused to various constructs. Additionally, the amino terminus of most of these proteins resides on the outside of the magnetosome, meaning that the primary amines are readily available to react with, for example, aldehyde terminated fluorescently labeled oligos. In some embodiments, the magnetotactic bacteria are engineered to express one or more of the magnetosome membrane proteins such as MamC as a fusion construct with a coupling agent such as streptavidin, facilitating attachment of detectable labels and/or oligonucleotides to the magnetosome using a binding partner such as biotin.

In some embodiments, the methods provided herein comprise detecting a single detectably labeled magnetic particle bound to a single target nucleic acid molecule. In some embodiments, two or more detectably labeled magnetic particles are bound to a single target nucleic acid molecule. Indeed, by titrating down the number of oligonucleotides on each detectably labeled magnetic particle, in some aspects, the particles can be designed to ensure that one particle will not bind and occupy all available intermediate probes on the target nucleic acid (e.g., rolling circle amplification product), permitting more than one detectably labeled magnetic particle to bind each target nucleic acid molecule (e.g., rolling circle amplification product). In some embodiments, occupying the remaining available coupling agents on the magnetic particle with fluorophores produces maximum signal.

In some aspects, provided herein are methods comprising in situ assays using microscopy as a readout, e.g., nucleic acid sequencing, hybridization, or other detection or determination methods involving an optical readout, wherein a detectably labeled magnetic particle is used to detect nucleic acid targets. In some embodiments, contacting the biological sample with the detectably labeled magnetic particle comprises contacting the biological sample with a pool of detectably labeled magnetic particles comprising different detectable labels. In some aspects, detection or detection or determination of a sequence of one, two, three, four, five, or more nucleotides of a target nucleic acid is performed in situ in a cell in an intact tissue. In some embodiments, the detection is used to reveal the presence/absence, distribution, location, amount, level, expression, or activity of the one or more analytes in the sample. In some embodiments, the assay comprises detecting the presence or absence of an amplification product (e.g., RCA product). In some embodiments, the present disclosure provides methods for sensitive detection that additionally facilitate removal of the detectable label from the targets in situ, such as transcripts and/or DNA loci, e.g., for detecting and/or quantifying nucleic acids and/or proteins in cells, tissues, organs or organisms

### II. Detectably Labeled Magnetic Particle

In some aspects, provided herein are detectably labeled magnetic particles (e.g., for use in a method of analyzing a biological sample. In some embodiments, a detectably labeled magnetic particle provided herein comprises a magnetic core, a detectable label, and oligonucleotides comprising a reporter hybridization sequence. In some embodiments, a plurality of moieties for different functionalities are attached to the magnetic particle (e.g., moieties for detection and moieties for removal on a single magnetic particle). In some embodiments, the magnetic particle comprises a coating or a membrane (e.g., a lipid membrane) surrounding the magnetic core. In some cases, the coating or membrane comprises coupling agents for attachment of the detectable label(s) and/or the oligonucleotides. For instance, the magnetic particle can be coated with streptavidin to facilitate attachment of biotinylated detectable labels (such as biotin-conjugated fluorophores) and/or biotinylated oligonucleotides. In some embodiments, the detectable label is associated with the magnetic core via the oligonucleotides. For example, in some embodiments all or a subset of the oligonucleotides are dually labeled with a biotin (to facilitate attachment to the coupling agent in the magnetic particle coating or membrane) and a detectable label such as a fluorophore. In some embodiments, detectable labels are attached to the magnetic particle via the coupling agent and via conjugation to all or a subset of the oligonucleotides.

In some embodiments, the detectably labeled magnetic particle has a diameter of between about 5 and 150 nm. In some embodiments, the detectably labeled magnetic particle has a diameter of between about 5 and 100 nm. In some embodiments, the detectably labeled magnetic particle has a diameter of between about 5 and 10 nm. In some embodiments, the detectably labeled magnetic particle has a diameter of between about 10 and 20 nm. In some embodiments, the detectably labeled magnetic particle has a diameter of between about 20 and 50 nm. In some embodiments, the detectably labeled magnetic particle has a diameter of between about 50 and 100 nm. In some embodiments, the detectably labeled magnetic particles have an average diameter of about 10 nm. In some embodiments, the detectably labeled magnetic particles have an average diameter of about 50 nm. In some embodiments, the detectably labeled magnetic particles have an average diameter of about 100 nm. In some embodiments, the detectably labeled magnetic particle has a diameter of no more than 200 nm, no more than 150 nm, no more than 100 nm, or no more than 50 nm. In some embodiments, the detectably labeled magnetic particles have less than 25% variation in diameters. In some embodiments, the detectably labeled magnetic particles have less than 20% variation in diameters. In some embodiments, the detectably labeled magnetic particles have less than 15% variation in diameters. In some embodiments, the detectably labeled magnetic particles have less than 10% variation in diameters.

Exemplary aspects of the detectably labeled magnetic particles and components thereof are described in further detail in the sections below.

### A. Magnetic Core and Coating or Membrane

Provided herein are detectably labeled magnetic particles comprising a magnetic core. In some aspects, the magnetic particles provided herein exhibit unique physicochemical properties, such as superparamagnetism, high surface/volume ratio, strong magnetic response, and low toxicity, depending on their size and shape. Nanoscaled magnetite (Fe₃O₄) is a kind of magnetic functional nanomaterial, wherein the electrons can hop between Fe²⁺ and Fe³⁺ ions in the octahedral sites at room temperature, making it an important class of spintronics material. The special characteristics of magnetic particles make them suitable candidates for a wide variety of applications in different fields of application such as nanotechnology, bioenvironmental, physical medicine, and engineering. Iron oxide nanoparticles such as magnetite (Fe₃O₄) or its oxidized form of hematite (α-Fe₂O₃) are the most commonly used nanoparticles for biomedical applications.

Examples of magnetic particles include any particle (such as a nanoparticle) which gives rise to a response when it is subjected to a magnetic field. In some embodiments, the response of the magnetic particle to a magnetic field is: i), a non-zero magnetization or coercivity, ii) a coercivity or magnetization that increases in strength with increasing magnetic field strength, iii) a magnetic particle magnetic moment that gets coupled with the magnetic field, and/or iv) a magnetic particle movement, optionally wherein the magnetic particle movement is induced when the magnetic field is non-uniform spatially. In some embodiments, the magnetic core of the magnetic particle includes ferromagnetic, ferrimagnetic, paramagnetic, superparamagnetic and/or diamagnetic materials. Non-limiting suitable examples can include: i) Fe₂O₃, Fe₃O₄, Fe₂O₄, FeₓPt_{y}, CoₓPt_{y}, MnFeₓO_{y}, CoFeₓO_{y}, NiFeₓO_{y}, CuFeₓO_{y}, ZaFeₓO_{y}, and CdFeₓO_{y}, wherein x and y are optionally between 1 and 6, depending on the method of synthesis used, and/or ii) nanoparticles comprising a magnetic material, preferentially predominantly, such as Fe, Pt, Au, Ag, Mg, Zn, Ni, or Si. In some embodiments the magnetic core comprises a material that is magnetic in the absence of application of an external magnetic field or in the presence of a magnetic field of strength lower than 1 mT, such as iron or iron oxide. In some embodiments, the magnetic core comprises a material that is magnetic in the presence of an external strength of strength preferentially higher than 10⁻⁵, 10⁻³, 1 or 10 mT, such as those composed of gold or silver.

In some embodiments, the magnetic core comprises one or more metal oxides. In some embodiments, the magnetic core comprises one or more iron oxides such as magnetite (Fe₃O₄) or maghemite (Fe₃O₃) or mixed phases resulting therefrom. In some embodiments, magnetic core contains portions of other bivalent or trivalent metal ions, such as for instance Ca²⁺, Ba²⁺, Zn²⁺, Co²⁺, Co³⁺, Cr³⁺, Ti³⁺, Mo²⁺, Mn²⁺, and Cu²⁺. In some embodiments, the magnetic core comprises a superparamagnetic iron oxide.

In some embodiments, the magnetic core comprises a ferromagnetic, ferrimagnetic, or paramagnetic material. In some embodiments, the magnetic core comprises a ferromagnetic material. Ferromagnetic materials may be strongly susceptible to magnetic fields and capable of retaining magnetic properties when the field is removed. Ferromagnetic materials include, but are not limited to, iron, cobalt, nickel, alloys thereof, and combinations thereof. Other ferromagnetic rare earth metals or alloys thereof can also be used to make the magnetic particles. In some embodiments, the magnetic core comprises a paramagnetic material. In some embodiments, when exposed to a magnetic field, the magnetic particles, depending on the magnetic properties, experience an alignment and they move corresponding to physical magnetic field gradients and react to temporal changes in the external magnetic field.

In some embodiments, the magnetic core has a diameter of between about 5 and 150 nm. In some embodiments, the magnetic core has a diameter of between about 5 and 100 nm. In some embodiments, the magnetic core has a diameter of between about 5 and 10 nm. In some embodiments, the magnetic core has a diameter of between about 10 and 20 nm. In some embodiments, the magnetic core has a diameter of between about 20 and 50 nm. In some embodiments, the magnetic core has a diameter of between about 50 and 100 nm.

In some embodiments, the magnetic cores of the detectably labeled magnetic particles have an average diameter of about 10 nm. In some embodiments, the magnetic cores of the detectably labeled magnetic particles have an average diameter of about 50 nm. In some embodiments, the magnetic cores of the detectably labeled magnetic particles have an average diameter of about 100 nm. In some embodiments, the magnetic cores of the detectably labeled magnetic particles have an average diameter of no more than 200 nm, no more than 150 nm, no more than 100 nm, or no more than 50 nm. In some embodiments, the magnetic cores of the detectably labeled magnetic particles have less than 10% variation in diameters.

In some embodiments, the magnetic particle comprises a coating or membrane (e.g., a lipid membrane). In some embodiments, the magnetic particle comprises a magnetic core with a coating. In some cases, the coating fully or partly surrounds the magnetic core. In some embodiments, the magnetic particle coating comprises or is linked to a coupling agent, such as streptavidin. In some embodiments, the coating comprises an amphiphilic polymer. In some embodiments, the coating comprises a water soluble material. In some embodiments, the coating comprises one or more layers. In some embodiments, the coating comprises a layer of oleic acid. In some embodiments, the coating comprises a layer of an amphiphilic polymer. In some embodiments, the coupling agent in or attached to the coating facilitates attachment of the detectable label and/or oligonucleotide molecules to the magnetic particle.

In some embodiments, the magnetic particle comprises a magnetic core surrounded fully or partly by a membrane. In some embodiments, a magnetosome comprises a magnetic core and membrane. In some embodiments, the membrane stabilizes the magnetic core and/or prevents or reduces the aggregation or agglomeration of the magnetic cores of a plurality of detectable magnetic particles.

### B. Oligonucleotides and Oligonucleotide Attachment

In some embodiments, the magnetic particle comprises oligonucleotides comprising a reporter hybridization sequence. In some embodiments, the oligonucleotides is bound to the magnetic particle through a coupling agent. In some embodiments, the coupling agent is a biotinylated lipid. In some embodiments, the coupling agent is a biotin-modified protein in the coating or lipid membrane. In some embodiments, the oligonucleotides comprising the reporter hybridization sequence are directly or indirectly attached to avidin, streptavidin, or neutravidin. In some embodiments, the coupling agent is or is fused to a protein in the protein coating or lipid membrane. In some embodiments, the coupling agent is selected from the group consisting of avidin, streptavidin, and neutravidin. In some embodiments, the 5′ end of the oligonucleotide is attached to the coupling agent. In some embodiments, the 3′ end of the oligonucleotide is attached to the coupling agent.

In some embodiments, at least 100-1000 oligonucleotides are coupled to the coupling agent molecules on the detectably labeled magnetic particle. In some embodiments, at least 100 oligonucleotides are coupled to the coupling agent molecules on the detectably labeled magnetic particle. In some embodiments, at least 500 oligonucleotides are coupled to the coupling agent molecules on the detectably labeled magnetic particle. In some embodiments, at least 1000 oligonucleotides are coupled to the coupling agent molecules on the detectably labeled magnetic particle. In some embodiments, the oligonucleotides are coupled to the detectably labeled magnetic particle via their 5′ ends. In some embodiments, the oligonucleotides are coupled to the detectably labeled magnetic particle via their 3′ ends.

In some embodiments, the oligonucleotides comprising the reporter hybridization sequence are biotinylated. In some embodiments, the oligonucleotides are detectably labeled. In some embodiments, the oligonucleotides attached to the magnetic particle is detectably labeled. In some embodiments, the methods include contacting the detectably labeled oligonucleotide with a reporter hybridization sequence complementary to the sequence of one or more of the oligonucleotides. In some embodiments, the reporter hybridization sequence is complementary to the reporter sequence of the intermediate probes, which corresponds to the detectable label in the detectably labeled magnetic particle. In some embodiments, the magnetic particle hybridizes to the reporter hybridization with complementarity to the sequence of one or more target nucleic acids sequence via one or more of the oligonucleotide molecules.

In some embodiments the reporter hybridization sequence is between about 10 and about 50 nucleotides in length, optionally between about 15 and about 25, e.g., about 20, nucleotides in length. In some embodiments, the linker sequence is between about 1 and about 10 nucleotides in length, optionally between about 1 and about 5 nucleotides in length, e.g., about 2 nucleotides in length.

In some embodiments, the oligonucleotide molecules individually comprise one or more crosslinkable nucleotides. In some embodiments, the method comprises crosslinking the hybridized reporter hybridization sequences to the intermediate probes before removing the detectably labeled magnetic particle. In some embodiments, the method provided therein comprises a crosslinkable moiety for interstrand crosslinking between the reporter hybridization sequence and the oligonucleotide. The hybridization region of the oligonucleotide may comprise one or more crosslinkable moieties (e.g., photoreactive nucleotide residues). In some embodiments, crosslinking is performed to form an interstrand crosslink between the oligonucleotide bound to the magnetic particle and the intermediate probe with complementarity to one or more target nucleic acids. In some embodiments, the crosslinking occurs in the hybridization region of the oligonucleotide bound to the magnetic particle. In some embodiments, crosslinking occurs upon activation by providing a stimulus. In some embodiments, the oligonucleotide bound to the magnetic particle is crosslinked to the intermediate probe with complementarity to one or more target nucleic acids via the one or more crosslinkable moieties in the hybridization region.

In some embodiments, the reporter hybridization sequence is between about 5 and about 50 nucleotides in length, such as between any of about 5 and about 30, about 5 and about 20, about 10 and about 25, and about 15 and about 30 nucleotides in length. In some embodiments, the hybridization region is between about 10 and about 15 nucleotides in length. In some embodiments, the hybridization region is at least about 5 nucleotides in length, such as at least any of about 10, 15, 20, 25, or 30 nucleotides in length.

In some of any of the embodiments herein, the reporter hybridization sequence is functionalized with a crosslinkable moiety. In some embodiments, the crosslinkable moiety is a photoreactive nucleotide. In some of any of the embodiments herein, the reporter hybridization sequence comprises two or more photoreactive nucleotides. In some of any of the embodiments herein, the hybridization region comprises three, four, five or more photoreactive nucleotides. In some of any of the embodiments herein, the hybridization region comprises about 2, 5, 10, 15, 20, 25, or 30 photoreactive nucleotides, or any range in between. Any one or more of the photoreactive nucleotides can be ^{CNV}D, ^{CNV}K, or any other suitable photoreactive nucleotides. In some embodiments, functionalization of the reporter hybridization sequence with a crosslinkable moiety facilitates removal of the intermediate probes hybridized to oligonucleotides of the detectably labeled magnetic particle by crosslinking the hybridized intermediate probes to the oligonucleotides and applying a magnetic field to the biological sample. Suitable crosslinkable moieties and methods of crosslinking are described in more detail in Section IV.

### C. Detectable Label

In some aspects, the provided methods comprise imaging a detectably labeled probe bound directly or indirectly to the magnetic particle and detecting the detectable label. In some aspects, the provided methods comprise imaging a detectably labeled magnetic particle and detecting the detectable label. In some embodiments, the detectable label is directly detectable by itself (e.g., radioisotope labels or fluorescent labels) or, in some embodiments (e.g., in the case of an enzymatic label), the detectable label is indirectly detectable, e.g., by catalyzing chemical alterations of a substrate compound or composition, which substrate compound or composition is directly detectable. In some embodiments, the detectably labeled magnetic particle comprises a detectable label that is measured and quantitated. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease. In some embodiments, the detectable label is a fluorescent protein. In some embodiments, the protein is green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

In some embodiments, the magnetic particle comprises a lipid membrane, and the fluorescent protein is fused to a protein in the lipid membrane. In some embodiments, attachment of the fluorescent protein to the magnetic particle comprises a coupling agent. In some embodiments, the coupling agent is a biotinylated lipid. In some embodiments, the coupling agent is a biotin-modified protein in the coating or lipid membrane. In some embodiments, the coupling agent is or is fused to a protein in the protein coating or lipid membrane. In some embodiments, the coupling agent is selected from the group consisting of avidin, streptavidin, and neutravidin. In some embodiments, the detectable label is a biotinylated fluorophore. In some embodiments, the magnetic particle is detectably labeled by at least a subset of the coupling agent molecules being coupled with a detectable label. In some embodiments, the detectably labeled magnetic particle comprises at least 50 fluorophores. In some embodiments, the detectably labeled magnetic particle comprises at least 500 fluorophores. In some embodiments, the detectably labeled magnetic particle comprises at least 1000 fluorophores.

In some embodiments, the detectably labeled magnetic particle comprises at least or at least about any of 10, 20, 50, 100, 200, 300, or 500 fluorophores (e.g., wherein the fluorophores are associated with the coupling agents in the magnetic particle coating or membrane and/or are attached to the oligonucleotides of the magnetic particle).

In some embodiments, a plurality of detectable labels are attached to the magnetic particle, oligonucleotide, or feature to be detected. For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™ Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18(5)), DIDS, Dil (DilC18(3)), DiO (DiOC18(3)), DiR (DilC18(7)), Di-4 ANEPPS, Di-8 ANEPPS, DM-NERF (4.5-6.5 pH), DsRed (Red Fluorescent Protein), EBFP, ECFP, EGFP, EFF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBFAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, EDS 751 (+DNA), EDS 751 (+RNA), FOFO™-1/FO-PRO™-1, Fucifer Yellow, FysoSensor™ Blue (pH 5), FysoSensor™ Green (pH 5), FysoSensor™ Yellow/Blue (pH 4.2), FysoTracker® Green, FysoTracker® Red, FysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO -PRO™-_, pQpO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO RholOl, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

### III. Methods of Making Magnetic Particles

In some aspects, provided herein is a method of making a detectably labeled magnetic particle comprising a magnetic core, a detectable label, and oligonucleotides comprising a reporter hybridization sequence.

In some embodiments, the magnetic core is produced through artificial means. Generally these methods for the synthesis of magnetic particles are categorized into two types of approaches: top-down and bottom-up approaches. Top-down approaches are used to prepare magnetic particles by breaking down bulk materials into nano-sized particles, such as ball milling, laser ablation, and spark ablation. Bottom-up approaches synthesize magnetic particles from nucleation and growth of atoms, such as physical methods like gel permeation chromatography, wet-chemical methods like co-precipitation, thermal decomposition, and sol-gel method. The spark ablation method is a top-down aerosol method for generating nanoparticles of mixed metals, which is environmentally friendly, scalable, and reasonably cheap. The method is based on two opposing electrodes that are charged until a spark arises. This spark hits the electrodes, and the material is ablated and transported away by a carrier gas flow. Due to the fast quenching of the metallic vapor, nucleation and alloy formation can occur at an early stage enabling formation of nanoalloys consisting of elements that are even immiscible in bulk. In some embodiments, small sub-10 nm primary particles are first formed and then transformed into larger agglomerates as they collide. In some embodiments, the magnetic core is produced by hydrothermal path, green synthesis, co-precipitation, laser ablation, or plasma spark ablation of iron oxides. In some embodiments, the magnetic core is produced by plasma spark ablation of iron oxides. In some embodiments, the magnetic core produced through artificial means is coated with a substance comprising a coupling agent selected from the group consisting of avidin, streptavidin, and neutravidin.

Iron oxide crystallites can be produced as metal oxide particles by a variety of methods, for instance by sintering at high temperatures with subsequent mechanical comminution, cluster formation under vacuum conditions, or wet chemical synthesis from solutions. The magnetic core can be coated with a polymer or membrane according to any suitable technique. In some embodiments, the coating or membrane comprises a coupling agent. In some embodiments, the method comprises contacting the magnetic core comprising a coating or membrane including a coupling agent with a detectable label attached to a binding partner which binds to the coupling agent, thereby attaching the detectable label to the magnetic particle. In some embodiments, the method comprises contacting the magnetic core comprising a coating or membrane including a coupling agent with an oligonucleotide comprising a binding partner which binds to the coupling agent, thereby attaching the oligonucleotide to the magnetic core.

In some embodiments, the coupling agent/binding partner pair comprises biotin and an avidin/streptavidin derivative. Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

In some embodiments, the detectably labeled magnetic particle is a modified magnetosome derived from magnetotactic bacteria (MTB). MTB are widespread, motile, diverse prokaryotes that biomineralize a unique organelle called the magnetosome. The magnetosome is specialized organelle comprising of a lipid-bilayer membrane that houses a crystal of the magnetic mineral magnetite (Fe₃O₄) or greigite (Fe₃S₄). In vitro synthetic magnetosomes have been produced by the chemical and genetic engineering methods, which provide the basis for the production and application of magnetosomes. However, the biosynthesis of magnetosomes has many advantages compared to a typical artificial magnetic nanoparticle. For example, the biological sources make it biocompatible, safe and superparamagnetic. Furthermore, the magnetosome is synthesized under strict genetic control with uniform shape, size, dispersion and chemical composition. The size of a magnetosome is normally between 35-120 nm, which is in a stable single magnetic domain. The magnetite particle distributions in the magnetosome typically are 35 to 120 nm.

In some embodiments, a magnetosome comprises a magnetic core (e.g., comprising any of the materials described in Section II.A above) surrounded fully or partly by a membrane. The membrane can be a synthetic membrane, e.g., membrane assembled in vitro and that is not synthesized by an organism. In some embodiments, the magnetosome membrane is a membrane synthesized by a living organism such as a magnetotactic microorganism. In some embodiments, the membrane is a membrane synthesized by a microorganism comprising lipids, lipopolysaccharide, endotoxins, and/or proteins or membrane-associated polypeptides produced by said microorganism. In some embodiments, the magnetosome magnetic core comprises iron oxide nanoparticles made of magnetite (Fe₃O₄), iron sulfide (greigite or Fe₃S₄) or mixtures thereof. In some embodiments, the magnetite becomes oxidized to maghemite. In some embodiments, the magnetosome magnetic core comprises a mixture of magnetite and maghemite.

In some embodiments, the magnetosome is produced by any organism that is capable of synthetizing metal-rich nanoparticle. In some embodiments, the magnetosome is produced by a magnetotactic microorganism (e.g., magnetotactic bacteria). In some embodiments, the magnetotactic microorganism is any organism which is capable of synthesizing iron-rich particles. In some instances, the presence of the iron-rich particles in the microorganism provides the microorganism with a permanent dipole moment or ferromagnetic or ferromagnetic properties, which can enable these microorganisms to orientate in a preferred direction, such as parallel to the geomagnetic field. Suitable magnetotactic microorganisms that can be used to produce the magnetic particles disclosed herein include, without limitation, Nitrospira, Nitrospira moscoviensis, Magnetobacterium bavaricum, Desulfovibrio magneticus RS-1, Desulfovibrio desulfuricans, Geobacter metallireducens, δ-Protobacteria, MMP5, MMP₂, where MM designates magnetotactic many-celled prokaryote, magnetic cossus, MC-1, CS103, NKMC5, α-Protobacteria, Rhodospirillum rubrum, Agrobacterium vitis, Magnetospirillum magnetotacticum MS-1, Magnetospirillum magneticum AMB-1, Magnetospirillum magneticum MGT-1, Magnetospirillum gryphiswaldense MSR-1, marine magnetic vibrio MV-1, Magnetospirillum gryphiswaldense MSR-1, Magnetospirillum magneticum AMB-1, Magnetospirillum magnetotacticum MS-1, Magnetospirillum magneticum strain MGT-1, magnetotactic coccus strain MC-1, Desulfovibrio magneticus RS-1 and anaerobic vibrio strains MV-1, MV-2 and MV-4. Suitable methods for determining whether a microorganism is magnetotactic include, without limitation: i) determination of the total content of iron, preferentially crystallized iron, inside these microorganisms, for example by atom absorption spectrophotometry as described by Suzuki T. et al (FEBS Lett., 2007, 581: 3443-3448) or Inductive coupled plasma mass spectrometry or iron dosage, ii) determination of the value of the C_{mag} parameter using light scattering technique (Schuler D. et al FEMS Microbiology Lett., 1995, 132: 139-145), iii) imaging these microorganisms by transmission electron microscopy as described by Qi et al. (PLoS One. 2012; 7(1):e29572).

In some aspects, provided herein is a method of producing a modified magnetosome comprising a detectable label and oligonucleotides. In some embodiments, a magnetosome is provided as described above via artificial means or by isolating a magnetosome from a living organism. In some embodiments, the detectable label and/or oligonucleotides are then attached to the magnetosome. In some embodiments, the magnetosome membrane (e.g., a synthetic membrane) comprises a lipid attached to a coupling agent (e.g., a biotinylated lipid such as biotin-DPPE). In some embodiments, a coupling agent is attached to proteins in the magnetosome membrane (e.g., using NHS-biotin).

In some embodiments, the magnetosome is a modified magnetosome derived from an engineered magnetotactic organism. In some embodiments, one or more magnetosome membrane proteins is operably linked to a coupling agent such as streptavidin. In some cases, the magnetotactic microorganism is engineered to express a magnetosome membrane protein operably linked to the coupling agent. The genes encoding magnetosome proteins are well characterized and are conserved in the genomes of magnetotactic bacteria in the form of a genomic island-like structure that is specifically referred to as the magnetosome island (MAI). Based on recent molecular analyses in the genetically tractable model organisms of magnetotactic bacteria Magnetospirillum magneticum AMB-1 and Magnetospirillum gryphiswaldense MSR-1, >30 proteins (magnetosome proteins) have been suggested to be involved in the process of magnetosome formation. In some embodiments, a subset of these proteins is/are engineered within a MTB to express the magnetosome membrane protein fused to the coupling agent. In some embodiments, the coupling agent is operably linked to a magnetosome membrane protein selected from the group consisting of: mamG, mamF, mamD, mamA, and mamC. In some embodiments, the magnetosome membrane protein is mamC protein. In some embodiments, the magnetosome is isolated from the magnetotactic bacteria by culturing the bacteria and then lysing the bacteria by sonication or other lysis techniques, followed by magnetic separation for purification of the magnetosome.

### IV. Methods of Analyzing a Biological Sample

In some aspects, provided herein are methods for analyzing a biological sample using detectably labeled magnetic particles. In some embodiments, provided herein is a method for analyzing a biological sample, comprising: a) contacting the biological sample with a detectably labeled magnetic particle, wherein the detectably labeled magnetic particle comprises: (i) a magnetic core, (ii) a detectable label, and (iii) oligonucleotides comprising a reporter hybridization sequence, wherein the reporter hybridization sequence is complementary to a reporter sequence present in a plurality of intermediate probes bound directly or indirectly to a target nucleic acid molecule in the biological sample, whereby the magnetic particle hybridizes to the reporter sequence of one or more of the probes via one or more of the oligonucleotide molecules and is thereby associated with the target nucleic acid molecule; and b) detecting the detectably labeled magnetic particle at a location in the biological sample. In some embodiments, any number of cycles of probe and detectably labeled magnetic particle hybridization, detection, and removal are performed to identify the target nucleic acid or a target sequence (such as a barcode sequence) within the target nucleic acid.

In some aspects, provided herein are methods comprising in situ assays using microscopy as a readout, e.g., using microscopy as an optical readout to detect a detectably labeled magnetic particle. In some embodiments, provided herein are methods comprising sequential hybridization of detectably labeled magnetic particles to a target nucleic acid (e.g., via hybridization to an intermediate probe that binds to the target nucleic acid). In some embodiments, the methods comprise applying a magnetic field to the biological sample to facilitate removal of the detectably labeled magnetic particle. Methods of magnetic particle removal are described in further detail in Section IV.A below. In some embodiments, the method comprises crosslinking one or more oligonucleotides of the detectably labeled magnetic particle to one or more intermediate probes hybridized thereto, to facilitate removal of the intermediate probes with the detectably labeled magnetic particle.

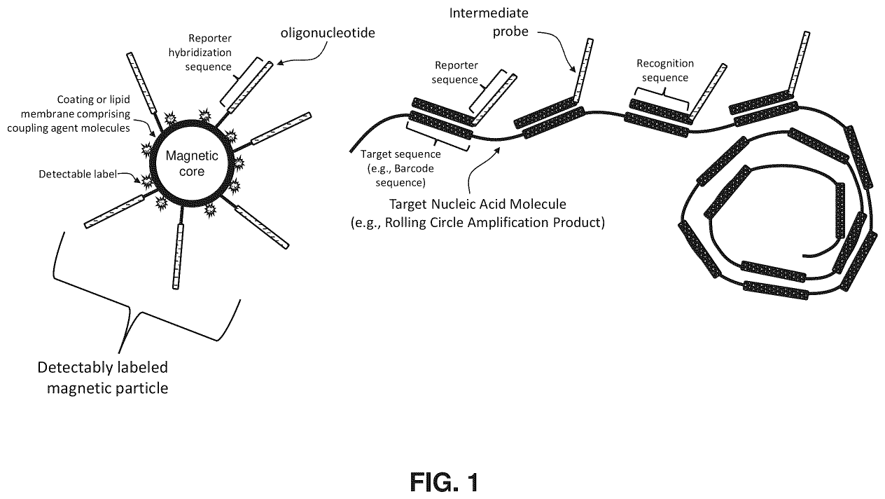

An example of a detectably labeled magnetic particle is illustrated in FIG. 1. As shown in FIG. 1, the detectably labeled magnetic particle comprises a magnetic core, oligonucleotides comprising a reporter hybridization sequence, and a detectable label. In the example depicted in FIG. 1, the magnetic particle comprises a coating or lipid membrane comprising coupling agent molecules for attachment of detectable labels such as fluorophores and/or oligonucleotides to the surface of the magnetic particle. Although the detectable label illustrated in the figure is attached to the coupling agent molecules in the coating or lipid membrane, a detectable label can additionally or alternatively be attached to the oligonucleotides. As shown in FIG. 1, the oligonucleotides comprise a reporter hybridization sequence. In some embodiments, the reporter hybridization sequence corresponds to the detectable label.

The reporter hybridization sequence can be complementary to a reporter sequence in a plurality of intermediate probes that hybridize to a target nucleic acid molecule such as a rolling circle amplification product, an endogenous analyte such as an RNA molecule, or a product such as a cDNA. In some embodiments, the intermediate probes comprise a recognition sequence complementary to a barcode sequence in the target nucleic acid product, and a reporter sequence complementary to the reporter hybridization sequence. In some embodiments, the intermediate probes comprise recognition sequences complementary to a plurality of target sequences in a target nucleic acid molecule such as an RNA (e.g., 20, 30, 40, or more probes can be designed to tile an RNA molecule). In some embodiments, the intermediate probes comprise a recognition sequence complementary to a barcode sequence in a plurality of primary encoding probes hybridized to an RNA analyte (e.g., 20, 30, 40, or more encoding probes can be designed to tile an RNA molecule). Various exemplary target nucleic acid molecules are described in Section IV.B below.

In some aspects, the provided methods involve analyzing, e.g., detecting or determining, one or more sequences present in the probes or probe sets or products thereof (e.g., rolling circle amplification products thereof). In some embodiments, the detecting is performed at one or more locations in the biological sample. In some embodiments, the locations are the locations of RNA transcripts in the biological sample. In some embodiments, the locations are the locations at which one or more primary probes or probe sets hybridize to the RNA transcripts in the biological sample, and are optionally ligated and amplified by rolling circle amplification to generate a rolling circle amplification molecule that is a target nucleic acid molecule for the intermediate probes.

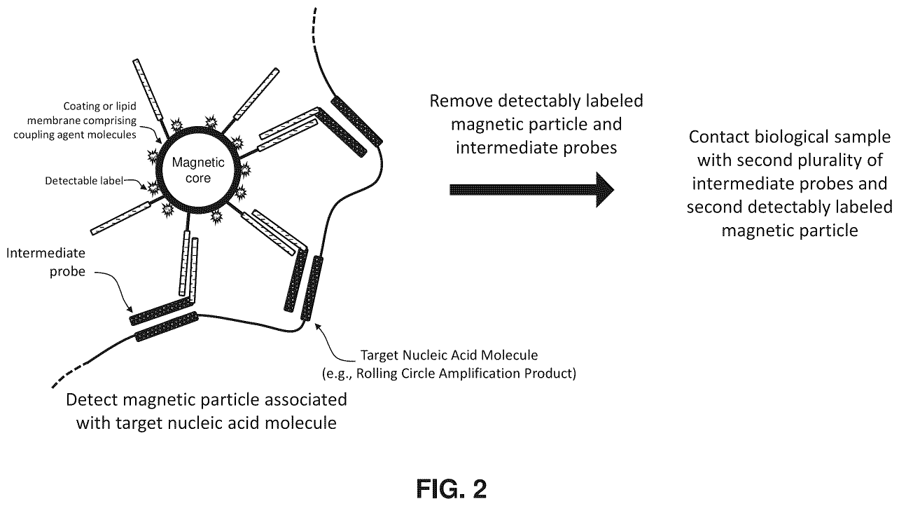

In some embodiments, the methods provided herein comprise detecting a detectably labeled magnetic particle bound in a complex with a target nucleic acid molecule (e.g., as illustrated in FIG. 2). As shown in FIG. 2, in some embodiments, multiple oligonucleotides of the detectably labeled magnetic particle hybridize to multiple intermediate probes bound to the target nucleic acid molecule. In some embodiments, the hybridization is cooperative, in that one or more initial hybridizations between one or more oligonucleotides of the detectably labeled magnetic particle and one or more intermediate probes brings one or more other oligonucleotides on the surface of the detectably labeled magnetic particle into closer proximity with one or more other intermediate probes bound to the target nucleic acid molecule, facilitating hybridization of the one or more other oligonucleotides to the one or more other intermediate probes. In some embodiments, hybridization of multiple oligonucleotides of the detectably labeled magnetic particle to multiple intermediate probes stabilizes association of the magnetic particle with the target nucleic acid.

As shown in FIG. 2, in some embodiments, after detecting the detectably labeled magnetic particle, the method comprises removing the detectably labeled magnetic particle and intermediate probes, and contacting the biological sample with a second plurality of intermediate probes and second detectably labeled magnetic particle. Thus, sequential cycles of hybridization of detectably labeled magnetic particles can be used to decode a barcode sequence and/or identify a target nucleic acid molecule.

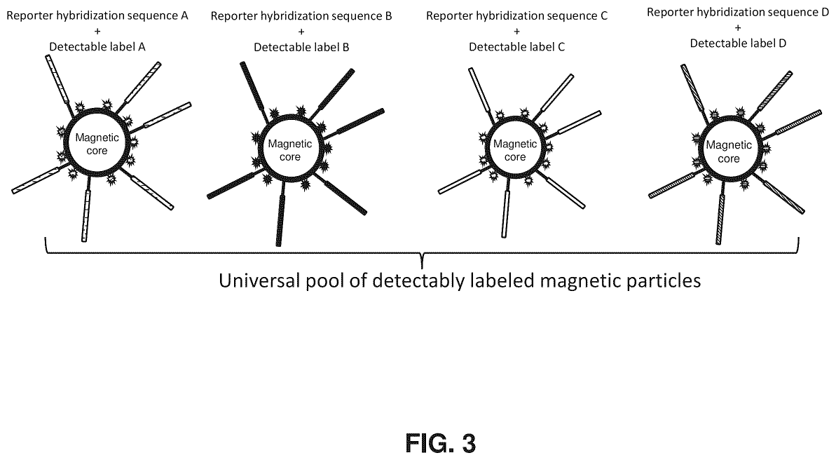

In some embodiments, detection of the barcode sequences is performed by sequential hybridization of intermediate probes to the barcode sequences or complements thereof and detecting complexes formed by the detectably labeled magnetic particles, the intermediate probes, and the barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled magnetic particles (e.g., as depicted in FIG. 3). In some cases, the sequences of signal codes are fluorophore sequences assigned to the corresponding barcode sequences or complements thereof. In some embodiments, the detectably labeled magnetic particles are fluorescently labeled. In some embodiments, the barcode sequence or complement thereof is performed by sequential probe hybridization as described in US 2021/0340618, the content of which is herein incorporated by reference in its entirety.

In any of the embodiments herein, the detecting step can comprise contacting the biological sample with one or more detectably labeled magnetic particles that directly or indirectly hybridize to the barcode sequences or complements thereof (e.g., in amplification products generated using the probes or probe sets), and dehybridizing the one or more detectably labeled magnetic particles. In some embodiments, the dehybridizing comprises applying a magnetic field to the biological sample. In any of the embodiments herein, the contacting and dehybridizing steps can be repeated with the one or more detectably labeled magnetic particles and/or one or more other detectably labeled magnetic particles that directly or indirectly hybridize to the barcode sequences or complements thereof. In some aspects, the method comprises sequential hybridization of detectably labeled magnetic particles to create a spatiotemporal signal signature or code that identifies the analyte.

In any of the embodiments herein, the detecting step can comprise contacting the biological sample with one or more first detectably labeled magnetic particles that directly hybridize to the intermediate probes associated with a plurality of different target nucleic acids. In some instances, the detecting step can comprise contacting the biological sample with one or more first detectably labeled magnetic particles that indirectly hybridize to the plurality of intermediate probes. In any of the embodiments herein, the detecting step can comprise contacting the biological sample with one or more first detectably labeled magnetic particles that directly or indirectly hybridize to the plurality of intermediate probes.

In any of the embodiments herein, the detecting step can comprise contacting the biological sample with one or more intermediate probes that directly or indirectly hybridize to the barcode sequences or complements thereof of the target nucleic acid molecule (e.g., of the plurality of probes or probe sets or rolling circle amplification product generated using the plurality of probes or probe sets), wherein the one or more intermediate probes are detectable using one or more detectably labeled magnetic particles. In any of the embodiments herein, the detecting step can further comprise dehybridizing the one or more intermediate probes and/or the one or more detectably labeled magnetic particles from the barcode sequences or complements thereof (e.g., of the plurality of probes or probe sets or rolling circle amplification product generated using the plurality of probes or probe sets). In any of the embodiments herein, the contacting and dehybridizing steps can be repeated with the one or more intermediate probes, the one or more detectably labeled magnetic particles, one or more other intermediate probes, and/or one or more other detectably labeled magnetic particles.

In any of the embodiments herein, the method can comprise contacting the sample with a pool of detectably labeled magnetic particles. In some embodiments, the pool of detectably labeled magnetic particles comprises multiple different detectably labeled magnetic particles comprising different detectable labels (e.g., detectable labels A, B, C, and D). The oligonucleotides on each different detectably labeled magnetic particle can comprise a reporter hybridization sequence corresponding to the detectable label of the probe (e.g., reporter hybridization sequences A, B, C, and D, corresponding to detectable labels A, B, C, and D, respectively). An exemplary pool of detectably labeled magnetic particles is illustrated in FIG. 3. In some embodiments, the pool comprises 2, 3, 4, 5, 6, or more different species of detectably labeled magnetic particles (e.g., detectably labeled magnetic particles comprising different fluorophores). In some embodiments, the same pool of detectably labeled magnetic particles (a universal pool) is used contacted with the biological sample for multiple sequential cycles of hybridization. A larger panel of intermediate probes can be used to hybridize to a larger panel of target sequences (e.g., a panel of 50, 100, 150, or more different barcode sequences) while providing a number of distinct reporter sequences corresponding to the number of different detectably labeled magnetic particles in the universal pool. The panels of intermediate probes can be contacted with a biological sample in a pre-determined sequence to yield a sequence of signals from the detectable labels that identifies the target sequences (e.g., barcode sequences).

In some embodiments, the method comprises a) contacting the sample with a panel of first probes and a pool comprising multiple species of detectably labeled magnetic particles, wherein each species of detectably labeled magnetic particles comprises: (i) a magnetic core, (ii) a different detectable label, and (iii) oligonucleotides comprising a reporter hybridization sequence corresponding to the detectable label; wherein each of a plurality of the first probes comprises (i) a recognition sequence complementary to a target sequence in a target nucleic acid molecule of the plurality of target nucleic acid molecules and (ii) a reporter sequence complementary to one of the reporter hybridization sequences in the pool of detectably labeled magnetic particles, and b) imaging the sample to detect a first signal from a complex formed between a detectably labeled magnetic particle of the pool of detectably labeled magnetic particles, a first probe of the panel of first probes, and a target nucleic acid of the plurality of target nucleic acid molecules. In some embodiments, the method comprises c) removing the first probe and the detectably labeled magnetic particle from the target nucleic acid molecule; d) contacting the sample with a panel of second probes and the pool of detectably labeled magnetic particles, wherein each of a plurality of the second probes comprises (i) a recognition sequence complementary to a target sequence in a target nucleic acid molecule of the plurality of target nucleic acid molecules and (ii) a reporter sequence complementary to one of the reporter hybridization sequences in the pool of detectably labeled magnetic particles, and e) imaging the sample to detect a second signal from a complex formed between a detectably labeled magnetic particle of the pool of detectably labeled magnetic particles, a second probe of the panel of second probes, and the target nucleic acid. In some embodiments, the first signal and the second signal are part of a sequence of signals that identifies the target nucleic acid molecule. In some embodiments, the plurality of target nucleic acid molecules is a plurality of rolling circle amplification products produced in the biological sample. In some embodiments, the rolling circle amplification products are associated with nucleic acid or non-nucleic acid analytes in the biological sample. In some embodiments, the detectable labels are fluorescent proteins or fluorophores, and the sequence of signals is a sequence of fluorescent signals.

A target sequence for a detectably labeled magnetic particle or an intermediate probe disclosed herein may be comprised in or associated with any analyte disclose herein, including an endogenous analyte (e.g., a viral or cellular nucleic acid), a labeling agent, or a product of an endogenous analyte and/or a labeling agent.

In some aspects, one or more of the target sequences includes one or more barcode(s), e.g., at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In any of the preceding embodiments, the methods provided herein can include analyzing the barcodes by sequential hybridization and detection with a plurality of detectably labeled magnetic particles.

In some embodiments, in a method of decoding a barcode sequence by sequential binding and removal of detectably labeled magnetic particles, barcode sequences are detected for identification of other molecules including nucleic acid molecules (DNA or RNA) longer than the barcode sequences themselves, as opposed to direct sequencing of the longer nucleic acid molecules. In some embodiments, a N-mer barcode sequence comprises 4^{N} complexity given a sequencing read of N bases, and a much shorter sequence of detectable signals may be required for molecular identification compared to non-barcode methods such as direct sequencing. For example, 1024 molecular species may be identified using a 5-nucleotide barcode sequence (4⁵=1024), whereas 8 nucleotide barcodes can be used to identify up to 65,536 molecular species, a number greater than the total number of distinct genes in the human genome. In some embodiments, the barcode sequences contained in the probes or RCPs are detected, rather than endogenous sequences, which can be an efficient read-out in terms of information per cycle of hybridization and detection. Because the barcode sequences are pre-determined, they can also be designed to feature error detection and correction mechanisms, see, e.g., U.S. Pat. Pub. 20190055594 and U.S. Pat. Pub 20210164039, which are hereby incorporated by reference in their entirety.

In some embodiments, the detecting comprises a plurality of repeated cycles of hybridization and removal of intermediate probes and/or detectably labeled magnetic particles (e.g., detectably labeled magnetic particles, or intermediate probes that bind to detectably labeled magnetic particles) to a target nucleic acid such as an RNA, to a primary probe or probe set hybridized to the target nucleic acid, or to a rolling circle amplification product generated from the probe or probe set hybridized to the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization assays, or sequencing by hybridization).

In some embodiments, the detecting can comprise binding an intermediate probe directly or indirectly to the primary probe or probe set, binding a detectably labeled magnetic particle directly or indirectly to a detection region of the intermediate probe, and detecting a signal associated with the detectably labeled magnetic particle. In some embodiments, the method comprises detecting a rolling circle amplification product (RCP) generated using a circular or circularized primary probe or probe set as a template. In some embodiments, the method comprises detecting a rolling circle amplification product (RCP) generated using a circular or circularized probe or probe that binds to a primary probe or probe set as a template. In some embodiments, detecting the RCP comprises binding an intermediate probe directly or indirectly to the RCP, binding a detectably labeled magnetic particle directly or indirectly to a detection region of the intermediate probe, and detecting a signal associated with the detectably labeled magnetic particle. In some embodiments, the method can comprise performing one or more wash steps to remove unbound and/or nonspecifically bound intermediate probe molecules from the primary probes or the products of the primary probes.

In some embodiments, the detecting can comprise: detecting signals associated with one or more detectably labeled magnetic particles that are bound directly or indirectly (e.g., via one or more intermediate probes) to one or more to barcode regions or complements thereof in the primary probe or probe set or a product thereof (e.g., an RCP). In some embodiments, the detecting comprises detecting signals associated with detectably labeled magnetic particles that are hybridized to intermediate probes which are in turn hybridized to the barcode regions or complements thereof. In some embodiments, the detectably labeled magnetic particles are fluorescently labeled.

In some embodiments, the methods comprise detecting the sequence in all or a portion of a primary probe or probe set or an RCP, or detecting a sequence of the primary probe or probe set or RCP, such as one or more barcode sequences present in the primary probe or probe set or RCP. In some embodiments, the sequence of the RCP, or barcode thereof, is indicative of a sequence of the target nucleic acid to which the RCP is hybridized. In some embodiments, the analysis and/or sequence determination comprises detecting a sequence in all or a portion of the nucleic acid concatemer and/or in situ hybridization to the RCP.

In some aspects, the provided methods comprise imaging a detectably labeled magnetic particle bound directly or indirectly to the target nucleic acid and detecting the detectable label. In some embodiments, the detectably labeled magnetic particle comprises a detectable label that is measured and quantitated. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. Exemplary detectable labels are described in Section II.C above.

In some embodiments, the detectable label is a fluorescent label. Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

In some aspects, the analysis can be carried out at room temperature for best preservation of tissue morphology with low background noise and error reduction. In some embodiments, the analysis and comprises eliminating error accumulation as the sequential cycles of hybridization and detection proceed.

In some embodiments, the analysis and/or sequence determination involves washing to remove unbound polynucleotides and/or unbound detectably labeled magnetic particles, thereafter revealing a fluorescent product (e.g., comprising one or more bound detectably labeled magnetic particle) for imaging.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detectably labeled magnetic particle. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detectably labeled magnetic particle. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

### A. Magnetic Particle Removal

In some embodiments, the methods provided herein comprise removing the detectably labeled magnetic particle from the biological sample after detecting the detectably labeled magnetic particle. In some embodiments, removing the detectably labeled magnetic particle comprises applying a magnetic field to the biological sample. In some embodiments, the magnetic field is applied using an electromagnet. In some embodiments, the magnetic field is a weak magnetic field. In some embodiments, the magnetic field is between about 500 nT to about 100 μT. In some embodiments, the magnetic field is between about 500 nT to about 1 μT. In some embodiments, the magnetic field is between about 1 μT to about 100 μT. In some embodiments, the magnetic field is between about 25 μT to about 75 μT. In some embodiments, the magnetic field is applied to the biological sample during a wash step to remove the detectably labeled magnetic particle(s). Provided herein is a system comprising a source for generating a magnetic field, including an opto-fluidic instrument for analysis of a biological sample, wherein the opto-fluidic instrument is configured to apply the magnetic field to the biological sample.

In some embodiments, the method comprises removing the detectably labeled magnetic particle and removing the intermediate probes to allow for a second plurality of intermediate probes to bind directly or indirectly to the target nucleic acid molecule. In some embodiments, application of the magnetic field to the biological sample also facilitates easier and/or faster removal of the intermediate probes hybridized to the detectably labeled magnetic particle. In some embodiments, one or more oligonucleotides of the detectably labeled magnetic particle are crosslinked to one or more intermediate probes that are hybridized to the oligonucleotides, facilitating simultaneous removal of the detectably labeled magnetic particle and the crosslinked intermediate probes by application of a magnetic field and one or more washes.

In some embodiments, an oligonucleotide of a detectably labeled magnetic particle provided herein comprises a crosslinkable moiety for interstrand crosslinking between the oligonucleotide of the detectably labeled magnetic particle and an intermediate probe hybridized to the oligonucleotide. In some embodiments, the crosslinkable moiety is or is in a photoreactive nucleotide residue. In some embodiments, the reporter hybridization region of the oligonucleotide comprises one or more crosslinkable moieties (e.g., photoreactive nucleotide residues). In some embodiments, crosslinking is performed to form an interstrand crosslink between the oligonucleotide of the detectably labeled magnetic particle and the intermediate probe hybridized to the oligonucleotide. In some embodiments, crosslinking is performed to form an interstrand crosslink between a plurality of the oligonucleotides of the detectably labeled magnetic particle and the intermediate probes hybridized to the oligonucleotides. In some embodiments, the crosslinking occurs in the reporter hybridization sequence of the oligonucleotide(s). In some embodiments, the oligonucleotide is crosslinked to the intermediate probe upon activation by providing a stimulus. In some embodiments, the oligonucleotide is crosslinked to the intermediate probe via the one or more crosslinkable moieties in the hybridization region. The crosslinkable moiety or moieties may become photo-activated as described in below, in order to crosslink the oligonucleotide(s) to the intermediate probe(s) hybridized thereto in the biological sample.

In some embodiments, activation of the crosslinkable moiety is light driven and can be performed in aqueous solution. In some embodiments, crosslinking strands of nucleic acid molecules comprise at least one photo-reactive nucleobase. In some embodiments, the crosslinkable moiety is a photo-reactive nucleobase. In some embodiments, the photo-reactive nucleobase can be any modified nucleobase that is capable of forming a crosslink with another nucleobase in an opposite hybridized strand in the presence of light. In some embodiments, the photo-reactive nucleobase can be a modified pyrimidine or purine nucleobase. In some embodiments, the photo reactive nucleobase can comprise a vinyl, acrylate, N-hydroxysuccinimide, amine, carboxylate or thiol chemical group. In some embodiments, the photo-reactive nucleobase comprises a bromo-deoxyuridine. Exemplary photoreactive crosslinkable moieties and photoreactive nucleotides are described, for example, in Elskens and Madder RSC Chem. Biol., 2021, 2, 410-422, the content of which is herein incorporated by reference in its entirety.

In some embodiments, the crosslinkable moiety comprises a reactive chemical group that requires light activation to initiate crosslinking. In some embodiments, the chemical group comprises, for example, an aryl azide, azido-methyl-coumarin, benzophenone, anthraquinone, certain diazo compounds, diazirine, or a psoralen derivative.

In some embodiments, the crosslinkable moiety comprises a cyanovinylcarbazole moiety. In some embodiments, the crosslinkable moiety comprises a 3-cyanovinylcarbazole (^{CNV}K) nucleoside or 3-cyanovinylcarbazole modified D-threoninol (^{CNV}D). In some embodiments, the crosslinkable moiety comprises 3-cyanovinylcarbazole phosphoramidite. In some embodiments, the crosslinkable moiety comprises a pyranocarbazole. In some embodiments, the crosslinkable moiety comprises a pyranocarbazole (^{PC}X) modified nucleoside or a pyranocarbazole with a D-threoninol instead of a 2′-deoxyribose backbone (^{PCX}D). In some embodiments, the crosslinkable moiety comprises a psoralen or a coumarin. In some embodiments, the photoreactive nucleotides have been attached to the oligonucleotide via a linker (e.g., a disulfide linker). In some embodiments, the crosslinkable moiety is a photoreactive nucleotide comprising a universal base.

In some embodiments, the crosslinkable moiety is a pyranocarbazole (^{PC}X) modified nucleoside. The ^{PC}X crosslinking base displays high crosslinking efficiency with a thymine (T) base or a cytosine (C) base that is positioned adjacent to the base on the complementary strand and can be directly incorporated into the DNA hybridization domain itself as a base substitution. In some embodiments, a crosslinking reaction is performed using 400 nm wavelength of light and can be completed within about 10 seconds. In some embodiments, a crosslinking reaction is completed within 0.1, 0.25, 0.5, 1, 5, or 10 seconds. In some embodiments, a crosslinking reaction is completed within 0.5, 1, 5, 10, 20, 30, 40, 50, or 60 minutes. In some embodiments, a crosslinking reaction is completed within 0.5, 1, 2, 3, 4, or 5 minutes. In some embodiments, a crosslinking reaction has negligible effects on bases that neighbor the photoreactive nucleobase. Other photochemical nucleic acid crosslinking agents, including psoralen can also be used in combination with the photoreactive nucleobases disclosed herein. In some embodiments, a photo-induced crosslink is reversible. In some embodiments, a photo-induced crosslink is reversed. In some embodiments, a ^{PC}X crosslink can be reversed when exposed to 312305 nm UV light.

In some embodiments, one or more other photochemical nucleic acid crosslinking agents, including psoralen and psoralen derivatives (e.g., psoralen modified nucleosides) is/are used as crosslinkable moieties. Psoralen and psoralen derivatives can be light-activated with a UV-A of 365 nm. Psoralens react with nearby pyrimidine residues. A variety of nucleosides modified with psoralen or psoralen derivatives may be used. For example, click chemistry using a psoralen azide and a nucleosidic alkyne derivative can be used to generate a variety of photoreactive nucleotides. The psoralen can be connected to the nucleotide via a linker, such as a phosphoramidite. Exemplary psoralen derivatives comprising phosphoramidite include but are not limited to 6-[4′-(Hydroxymethyl)-4,5′,8-trimethylpsoralen]-hexyl-1-O-(2-cyanoethyl)-(N,N-diisopropyl)-phosphoramidite and 2-[4′-(hydroxymethyl)-4,5′,8-trimethylpsoralen]-ethyl-1-O-(2-cyanoethyl)-(N,N-diisopropyl)-phosphoramidite. In some embodiments, the psoralen or psoralen derivative is conjugated to position 5 of a uridine or pseudouridine (optionally via a linker). In some cases, the psoralen or psoralen derivative is conjugated to the 2′ position of a sugar ring of a uridine or pseudouridine (optionally via a linker). In some embodiments, the psoralen derivative can be an amine-reactive derivative, which can be conjugated to an amine-modified nucleotide (e.g., an aminoallyl uridine or pseudouridine nucleotide).

In some embodiments, a psoralen-crosslink (e.g., an interstrand crosslink between the oligonucleotide and the hybridized intermediate probe) can be reversed when exposed to 254 nm light. In some embodiments, the crosslinkable moiety comprises a C2′ psoralen modification. The crosslinkable moiety can comprise a 5′ psoralen derivative, and can be at 5′ end of the oligonucleotide. The structure of two exemplary psoralen-modified oligonucleotides (one 5′ modified nucleoside on the left, and one C2′ modified nucleoside on the right) are shown below:

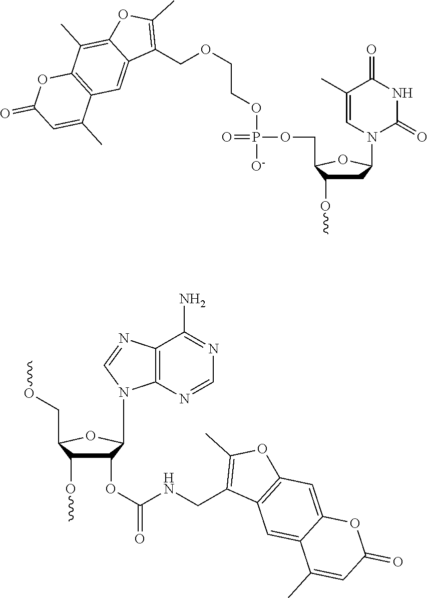

In some embodiments, the crosslinkable moiety is or is linked to a photoactivatable nucleotide, wherein the photoactivatable nucleotide is a universal base such as a pseudouridine modified with a photoreactive moiety (e.g. a psoralen).

In some embodiments, when the oligonucleotide comprises ^{CNV}K, rapid photo cross-linking to pyrimidines in the complementary strand (DNA or RNA) can be induced at one wavelength and rapid reversal of the cross-link is possible at a second wavelength if desired. Neither wavelength has the potential to cause significant DNA damage and neither interfere with the wavelengths used to excite the fluorophores used during subsequent analysis, such as decoding barcode sequences in situ. Once cross-linked, the UV melting temperature of the duplex may be raised by around 30° C./^{CNV}K moiety relative to the duplex before irradiation and inter-strand crosslinking. The structure of an exemplary 3-cyanovinylcarbazole phosphoramidite is shown below:

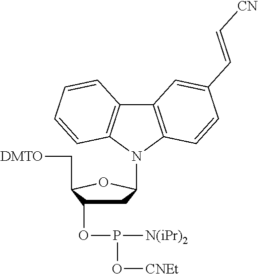

### 5′-O-(4,4′-Dimethoxytrityl)-1′-(3-cyanovinylcarbazol-9-yl)-2′-deoxy-β-D-ribofuranosyl-3′-[(2-cyanoethyl)-(N,N-diisopropyl)]-phosphoramidite

The ^{CNV}K Crosslinking base displays high crosslinking efficiency with a thymine (T) base that is positioned adjacent to the base on the opposite hybridized strand in the target nucleic acid (e.g., the complementary strand) (Ultrafast reversible photo-cross-linking reaction: toward in situ DNA manipulation. Org. Lett. 10, 3227-3230 (2008)) and can be directly incorporated into the DNA hybridization domain itself as a base substitution, as shown below in light-directed reaction between a ^{CNV}K base modification and a thymine base to produce a crosslinked nucleic acid.

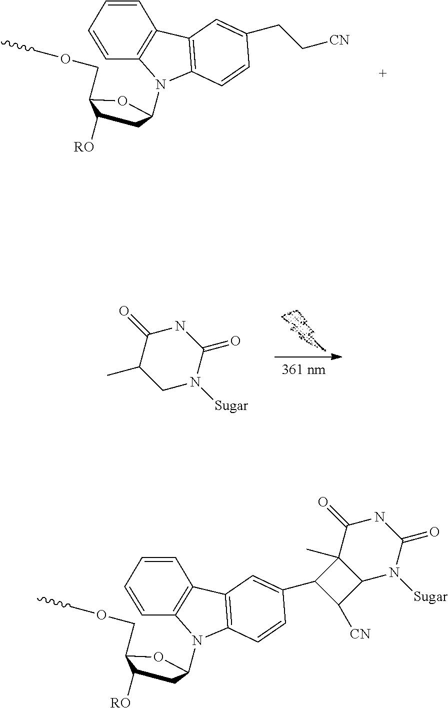

In some embodiments, a crosslinking reaction is performed using 365 nm wavelength of light and is completed within about 1 second. The crosslinking reaction can be performed using any wavelength of visible or ultraviolet light, which will depend on the crosslinking moiety selected. In some embodiments, a crosslinking reaction can is completed within 0.1, 0.25, 0.5, 1, 5, or 10 seconds. In some embodiments, a crosslinking reaction is completed within 20, 30, 40, 50, or 60 seconds. In some embodiments, the method comprises irradiating the biological sample with UV light, such as a 350-400 nm wavelength of light, for between 10 seconds and 10 minutes, between 10 seconds and 5 minutes, between 10 seconds and 2 minutes, between 10 seconds and 1 minute, between 30 seconds and 1 minute, or between 30 seconds and 5 minutes. In some embodiments, a crosslinking reaction is completed within 0.5, 1, 5, 10, 20, 30, 40, 50, or 60 minutes. In some embodiments, a crosslinking reaction has negligible effects on bases that neighbor the photoreactive nucleobase. In some embodiments, one or more other photochemical nucleic acid crosslinking agents, including psoralen and/or coumarin is or are used in combination with the photoreactive nucleobases disclosed herein.

In some embodiments, the crosslinkable moiety comprises a coumarin and the photoactivation comprises irradiating the biological sample using a 350 nm wavelength of light. In some embodiments, the crosslinkable moiety comprises a psoralen and the photoactivation comprises irradiating the biological sample using a 365 nm wavelength of light. In some embodiments, the crosslinkable moiety comprises a ^{CNV}K or ^{CNV}D and the photoactivation comprises irradiating the biological sample using a 365 nm wavelength of light. In some embodiments, the crosslinkable moiety comprises a ^{PC}X or ^{PCX}D and the photoactivation comprises irradiating the biological sample using a 400 nm wavelength of light. In some embodiments, the crosslinkable moiety comprises a diazirine and the photoactivation comprises irradiating the biological sample using a 365 nm wavelength of light. In some embodiments, the crosslinkable moiety comprises a thiouridine and the photoactivation comprises irradiating the biological sample using a 365 nm wavelength of light.

In some embodiments, a photo-induced crosslink is reversed. In some embodiments, a vinylcarbazole (e.g., ^{CNV}K ^{CNV}D, ^{PC}X, or ^{PCX}D) crosslink is reversed when exposed to 305 nm UV light. In some embodiments, a vinylcarbazole (e.g., ^{CNV}K ^{CNV}D, ^{PC}X, or ^{PCX}D) crosslink is reversed when exposed to 312 nm light. In some embodiments, a psoralen crosslink is reversed when exposed to 254 nm light. In some embodiments, a coumarin crosslink is reversed when exposed to 254 nm light.

In some embodiments, the crosslinkable moiety is a photoactivatable nucleotide comprising a coumarin and hybridizes to a thymine (T) base in the complementary strand. In some embodiments, the photoactivatable nucleotide comprises a psoralen and hybridizes to a C, T, or U base in the complementary strand. In some embodiments, the photoactivatable nucleotide comprises a vinylcarbanazole and hybridizes to a C, T, or U base in the complementary strand. In some embodiments, the photoactivatable nucleotide comprises a universal or random base.

In some embodiments, the crosslinkable moiety crosslinks to an adenine (A) nucleobase in the strand of the target nucleic acid hybridized to the oligonucleotide. In some embodiments, the crosslinkable moiety comprises a psoralen capable of crosslinking to an adenine in the hybridized nucleic acid strand. In some embodiments, the crosslinkable moiety comprises a 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite). In some embodiments, crosslinkable moiety comprises a 5′-Dimethoxytrityl-5-iodo-2′-deoxyUridine,3′-[(2-cyanoethyl)-(N,N-diisopropyl)]-phosphoramidite (5-I-dU-CE phosphoramidite).

The structure of an exemplary psoralen C2 phosphoramadite crosslinkable moiety is shown below:

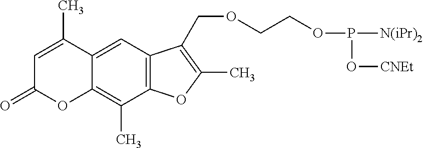

The structure of an exemplary 5′-Dimethoxytrityl-2′-deoxy-4-(2-cyanoethylthio)-Thymidine,3′-[(2-cyanoethyl)-(N,N-diisopropyl)]-phosphoramidite (4-Thio-dT-CE phosphoramidite) crosslinkable moiety is shown below:

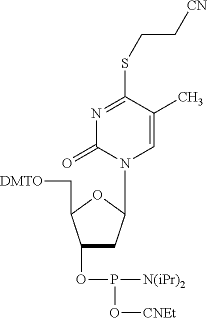

The structure of an exemplary 5′-Dimethoxytrityl-5-iodo-2′-deoxyUridine,3′-[(2-cyanoethyl)-(N,N-diisopropyl)]-phosphoramidite (5-I-dU-CE phosphoramidite) crosslinkable moiety is shown below:

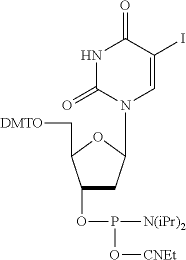

The photoreactive nucleotides may be photo-activated by UV light, such as a 350-400 nm wavelength of light, to photo-activate and crosslink the crosslinkable moiety of the hybridized oligonucleotide of the detectably labeled magnetic particle to the intermediate probe. In some embodiments, the crosslinkable moiety is crosslinked to the complementary strand at a 355 nm wavelength of light. In some embodiments, the purine bases of the target nucleic acid are unreactive to photo-activated crosslinking. In some embodiments, the pyrimidine bases of the complementary strand are reactive to photo-activated crosslinking. In some embodiments, the purine bases of the target nucleic acid are reactive to crosslinking (e.g., to a psoralen, 5-I-dU-CE, 4-Thio-dT-CE, or any other crosslinkable moiety configured to crosslink with nucleobases including adenine).

The photo-activated crosslinking step may be optimized to prevent DNA damage. In some embodiments, the photo-activated crosslinking does not cause significant DNA damage. In some embodiments, the photo-activated crosslinking between the oligonucleotide of the detectably labeled magnetic particle and an intermediate probe hybridized to the oligonucleotide increases the UV melting temperature of the duplex compared to prior to the crosslinking. In some embodiments, the UV melting temperature is increased by about 30° C. per photoreactive nucleotide in the hybridization region.

### B. Target Nucleic Acid Molecules and Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte is directly or indirectly detected using the detectably labeled magnetic particle(s) disclosed herein.

In some embodiments, the target nucleic acid molecule according to the methods disclosed herein is a nucleic acid analyte or is associated with an analyte in the biological sample. For example, a target nucleic acid molecule associated with a nucleic acid analyte can be a product of the analyte such as a cDNA produced from an RNA analyte in the biological sample. In some embodiments, the target nucleic acid molecule is a primary probe hybridized to an analyte or labeling agent in the biological sample, or is a product of a probe bound directly or indirectly to an analyte or labeling agent in the biological sample. In some embodiments, the target nucleic acid molecule is a rolling circle amplification product of a circular probe or circularizable probe or probe set bound directly or indirectly to an analyte or labeling agent in the biological sample.

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte is an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. In some embodiments, the RNA comprises circular RNA. In some embodiments, the RNA is a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, the plurality of intermediate probes bind to a target nucleic acid molecule in or associated with a labeling agent, so that the detectably labeled magnetic probe is associated with the labeling agent upon hybridization to one or more of the probes. In some embodiments, the target nucleic acid molecule is a rolling circle amplification product associated with the labeling agent. In some embodiments, the rolling circle amplification product is produced from a circular or circularizable probe or probe set that hybridizes to a reporter oligonucleotide in the labeling agent. In some embodiments, the method comprises circularizing the circularizable probe or probe set (e.g., by ligation). The circular or circularized probe can then be amplified by rolling circle amplification. In some embodiments, the rolling circle amplification is performed using a primer, or using the reporter oligonucleotide of the labeling agent as a primer. In some instances, where the reporter oligonucleotide of the labeling agent is used as a primer for rolling circle amplification, the rolling circle amplification product is attached to the labeling agent (via the reporter oligonucleotide incorporated into the RCA product). In some embodiments, the RCA product comprises multiple copies of a barcode sequence corresponding to the labeling agent, allowing for identification of an analyte bound by the labeling agent by analysis of the barcode sequence.

In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, the method comprises one or more post-fixing steps after contacting the sample with one or more labeling agents. In some embodiments, the method comprises one or more post-fixation steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample are subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### (a) Hybridization

In some embodiments, a product of an endogenous analyte and/or a labeling agent is a hybridization product comprising the pairing of substantially complementary or complementary nucleic acid sequences within two different molecules, one of which is the endogenous analyte or the labeling agent (e.g., reporter oligonucleotide attached thereto). The other molecule can be another endogenous molecule or another labeling agent such as a probe. In some cases, the hybridization is between a probe described herein and the target nucleic acid molecule. In some cases, the hybridization is between the oligonucleotide of the detectably labeled magnetic particle and an intermediate probe hybridized to the oligonucleotide. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes and probe sets can be hybridized to an endogenous analyte and/or a labeling agent and each probe may comprise one or more barcode sequences. Exemplary barcoded probes or probe sets may be based on a circularizable probe or probe set (e.g., padlock probe), a gapped padlock probe, a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary. In some embodiments, the target nucleic acid is a rolling circle amplification product produced using any suitable circular or circularizable barcoded probes or probe sets.

### (b) Ligation

In some embodiments, the target nucleic acid molecule analyzed using the detectably labeled magnetic particle(s) herein is a ligation product or an amplification product produced from a ligation product. In some embodiments, the ligation product is formed between two or more endogenous analytes. In some embodiments, the ligation product is formed between two or more labeling agents. In some embodiments, the ligation product is an intramolecular ligation of an endogenous analyte. In some embodiments, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, the target nucleic acid molecule is a probe or probe set capable of DNA-templated ligation, such as from a cDNA molecule, or an amplification (such as an RCA product) of such a probe or probe set. See, e.g., U.S. Pat. No. 8,551,710, which is hereby incorporated by reference in its entirety. In some embodiments, the target nucleic acid molecule is a probe or probe set capable of RNA-templated ligation, or an amplification (such as an RCA product) of such a probe or probe set. See, e.g., U.S. Pat. Pub. 2020/0224244 which is hereby incorporated by reference in its entirety. In some embodiments, the target nucleic acid molecule is a rolling circle amplification product produced using a SNAIL probe set. See, e.g., U.S. Pat. Pub. 20190055594, which is hereby incorporated by reference in its entirety. In some embodiments, the target nucleic acid molecule is a rolling circle amplification product produced in a multiplexed proximity ligation assay. See, e.g., U.S. Pat. Pub. 20140194311 which is hereby incorporated by reference in its entirety. In some embodiments, the target nucleic acid molecule is a rolling circle amplification product of a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, the target nucleic acid molecule is a rolling circle amplification product of a circular probe. In some embodiments, a circular probe can be directly or indirectly bound to a nucleic acid analyte or a labeling agent (e.g., a probe for detecting a nucleic acid or non-nucleic acid analyte). In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, e.g., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, circularizable probe or probe set (e.g., padlock probe), or target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some embodiments, the target nucleic acid molecule is a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. In some embodiments, a primer is a primer binding sequence. In some embodiments, a primer extension reaction is any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, the target nucleic acid molecule is a product of an endogenous analyte and/or a labeling agent. In some embodiments, the target nucleic acid molecule is an amplification product of one or more polynucleotides, for instance, a circular probe or circularizable probe or probe set. In some embodiments, the amplifying is achieved by performing rolling circle amplification (RCA). In some embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, amplification of the probes or probe sets is primed by the respective target RNAs. In some embodiments, the target RNAs are immobilized in the biological sample. In some embodiments, the target RNAs is cleaved by an enzyme (e.g., RNase H). In some embodiments, the target RNA is cleaved at a position downstream of the target sequences bound to the circularized probe or probe set. In some aspects, the methods disclosed herein allow targeting of RNase H activity to a particular region in a target RNA that is adjacent to or overlapping with a target sequence for a probe or probe set. For example, a nucleic acid oligonucleotide is designed to hybridize to a complementary oligonucleotide hybridization region in the target RNA. In some embodiments, a nucleic acid oligonucleotide is used to provide a DNA-RNA duplex for RNase H cleavage of the target RNA in the DNA-RNA duplex. In some embodiments, the oligonucleotide binds to the target RNA at a position that overlaps with the target sequence of the probe by about 1 to about 20 nucleotides or by about 8 to about 10 nucleotides. In some embodiments, the cleaved target RNA itself is then used to prime RCA of the circularized probe. In some cases, a plurality of nucleic acid oligonucleotides are used to perform target-primed RCA for a plurality of different target RNAs.

In any of the embodiments herein, the biological sample is contacted with the RNase H (and optionally with the nucleic acid oligonucleotide) before or during formation of the circularized probe or probe set. In some embodiments, the biological sample is contacted with the oligonucleotide and with the RNase H simultaneously or sequentially (in either order) before contacting the sample with the probes. In any of the embodiments herein, the biological sample is contacted with the RNase H (and optionally with the nucleic acid oligonucleotide) after formation of the circularized probe(s). In any of the embodiments herein, the RNase H comprises an RNase H1 and/or an RNAse H2. In some embodiments, RNase inactivating agents or inhibitors are added to the sample after cleaving the target RNA.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 Nov. 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:el 18, 2001; Dean et al. Genome Res. 1 1 :1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, US 2016/0024555, US 2018/0251833 and US 2017/0219465, all of which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template comprises the target analyte, or a part thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

### V. Compositions, Kits, and Systems

In some aspects, provided herein are compositions comprising any of the detectably labeled magnetic particles described in Section II above. In some embodiments, the composition comprises a pool of detectably labeled magnetic particles comprising at least four different species of detectably labeled magnetic particles, wherein each species of detectably labeled magnetic particles comprises: (i) a magnetic core, (ii) a different detectable label, and (iii) oligonucleotides comprising a reporter hybridization sequence corresponding to the detectable label. In some embodiments, the different detectable labels are different fluorophores.

Also provided herein are kits, for analyzing an analyte in a biological sample according to any of the methods described herein. In some embodiments, provided herein is a kit comprising one or more of the detectably labeled magnetic particles disclosed herein, such as any of the detectably labeled magnetic particles described in Section II above. In some embodiments, the kit comprises a plurality of detectably labeled magnetic particles.

In some aspects, provided herein is a kit comprising a) at least four different species of detectably labeled magnetic particles, wherein each species of detectably labeled magnetic particles comprises: (i) a magnetic core, (ii) a different detectable label, and (iii) oligonucleotides comprising a reporter hybridization sequence corresponding to the detectable label; and b) a sequential series of probe panels, wherein each panel comprises multiple probe species, and wherein each probe species comprises (i) a barcode recognition sequence complementary to a different barcode sequence, and (ii) a reporter sequence, which can be the same or different from the reporter sequence of a different probe species in the panel; wherein the reporter sequence is complementary to the reporter hybridization sequence of one of the species of detectably labeled magnetic particles. The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

Provided herein is a system for performing the methods described herein (e.g., for analyzing a biological sample using detectably labeled magnetic particles). Provided herein is a system comprising a source for generating a magnetic field for removing the detectably labeled magnetic particle from the biological sample after detecting the detectably labeled magnetic particle. In some embodiments, the system comprises a source for generating light for irradiating the biological sample (e.g., to photo-activate the crosslinkable moiety).

### VI. Biological Samples and Sample Preparation

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or circularizable probe or probe set (e.g., padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set (e.g., padlock probe).

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 m to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample is segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016 and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, is added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest can be selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte can be used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

A biological sample may comprise one or a plurality of analytes of interest. Methods for performing multiplexed assays to analyze two or more different analytes using the detectably labeled magnetic particles disclosed herein in a single biological sample are provided.

### VII. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VIII. Opto-Fluidic Instruments for Analysis of Biological Samples

Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., detectably labeled magnetic particles) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more analytical cycles (e.g., as described in Section IV). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

In some embodiments, the opto-fluidic instrument is configured to apply a magnetic field to the biological sample. In some embodiments, the opto-fluidic instrument is configured to apply a magnetic field to the biological sample during a wash step (e.g., for removal of magnetic particles from the biological sample). In some embodiments, the magnetic field is applied using an electromagnet. In some embodiments, the magnetic field is a weak magnetic field. In some embodiments, the magnetic field is between about 500 nT to about 100 μT. In some embodiments, the magnetic field is between about 500 nT to about 1 μT. In some embodiments, the magnetic field is between about 1 μT to about 100 μT. In some embodiments, the magnetic field is between about 25 μT to about 75 μT. In some embodiments, the magnetic field is applied to the biological sample during a wash step to remove the detectably labeled magnetic particle(s). Provided herein is a system comprising a source for generating a magnetic field. In some embodiments, the opto-fluidic instrument is configured to remove magnetic particles from a biological sample according to any of the methods described in Section IV.A.

It is to be noted that, although the above discussion relates to an opto-fluidic instrument that can be used for in situ target molecule detection via hybridization to a magnetic particle, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

### IX. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods. “Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Analyte Detection Using Modified Magnetosomes

This example describes an exemplary method for analyzing a biological sample using detectably labeled magnetic particles.

The biological sample (e.g., tissue sample) is fixed onto an optically transparent substrate such as a slide or coverslip and optionally permeabilized. Upon tissue fixation, mRNAs are targeted in situ by a panel of circularizable nucleic acid probes. The circularizable nucleic acid probes hybridize to the targeted mRNAs. Hybridized circularizable probes are then ligated to generate circularized nucleic acid probes at locations in the biological sample. Rolling circle amplification is performed using the circularized probes as templates to generate a plurality of rolling circle amplification products (RCPs).

The circularizable probes comprise barcode sequences corresponding to their respective target nucleic acid molecules (e.g., RNA analytes). Each of the RCPs comprises multiple copies of the complement of the barcode sequence, also referred to as a barcode sequence for simplicity. A sequence of signal codes (a fluorophore sequence) is assigned to each of the barcode sequences, and a sequential series of intermediate probe panels targeting the RCP barcode sequences is provided, wherein sequential hybridization of the intermediate probes to their respective barcode sequences yields sequential association of reporter sequences corresponding to the sequence of signal codes with the barcode sequence.

The sample is contacted with the first intermediate probe panel. A universal pool of detectably labeled magnetic particles is provided, comprising detectably labeled magnetic particles comprising four different detectable labels (e.g., as illustrated schematically in FIG. 3). The detectably labeled magnetic particles can comprise a magnetic core and a coating or lipid membrane comprising a coupling agent such as streptavidin for binding to a binding partner such as biotin, thereby attaching a plurality of molecules of a detectable label and oligonucleotides comprising a reporter hybridization sequence corresponding to the detectable label to the magnetic particle. The sample is contacted with the universal pool of detectably labeled magnetic particles. Under conditions optimized for hybridization of the intermediate probes to their respective barcode sequences, and of the detectably labeled magnetic probes to their corresponding reporter sequence in the intermediate probes, the sample is incubated for each hybridization step. The sample is then imaged to detect the detectably labeled magnetic probes. A weak magnetic field is applied to the sample during one or more wash steps to remove the detectably labeled magnetic probes and the first panel of intermediate probes.

The contacting, imaging, and removal can be repeated with a second panel of intermediate probes and the universal pool of detectably labeled magnetic particles, followed by a third panel of intermediate probes and the universal pool of detectably labeled magnetic particles, and so on until sufficient signal codes (e.g., a temporal signal signature or code of signals detected from cycles of bound detectably labeled magnetic particles) have been detected to decode the barcode sequences and identify the target mRNAs at the locations in the biological sample.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-88. (canceled)

89. A method for analyzing a biological sample, comprising:
a) contacting the biological sample with a detectably labeled magnetic particle, wherein the detectably labeled magnetic particle comprises:
(i) a magnetic core,
(ii) a detectable label, and
(iii) oligonucleotides comprising a reporter hybridization sequence,
wherein the reporter hybridization sequence is complementary to a reporter sequence present in a plurality of intermediate probes bound directly or indirectly to a target nucleic acid molecule in the biological sample, whereby the magnetic particle hybridizes to the reporter sequence of one or more of the intermediate probes via one or more of the oligonucleotide molecules and is thereby associated with the target nucleic acid molecule; and
b) detecting the detectably labeled magnetic particle at a location in the biological sample.

90. The method of claim 89, wherein the detectably labeled magnetic particle comprises a coating or lipid membrane comprising a plurality of coupling agent molecules, wherein at least a subset of the coupling agent molecules is coupled with a binding partner associated with the oligonucleotide molecules.

91. The method of claim 90, wherein the coupling agent is a biotinylated lipid or a biotin-modified protein in the coating or lipid membrane, wherein the binding partner associated with the oligonucleotide molecules and/or the binding partner associated with the detectable label comprises avidin, streptavidin, or neutravidin.

92. The method of claim 90, wherein the coupling agent is or is fused to a protein in the protein coating or lipid membrane.

93. The method of claim 90, wherein the coupling agent is selected from the group consisting of avidin, streptavidin, and neutravidin, and wherein the binding partner associated with the oligonucleotide molecules and/or the binding partner associated with the detectable label comprises biotin.

94. The method of claim 89, wherein the method comprises: c) removing the detectably labeled magnetic particle and the plurality of intermediate probes from the biological sample after detecting the detectably labeled magnetic particle b) by applying a magnetic field to the biological sample.

95. The method of claim 89, wherein the oligonucleotide molecules individually are functionalized with a crosslinkable moiety, and wherein the method comprises crosslinking the reporter hybridization sequences to the hybridized one or more intermediate probes before removing the detectably labeled magnetic particle.

96. The method of claim 94, wherein the method comprises:
d) binding a second plurality of intermediate probes comprising a second reporter sequence directly or indirectly to the target nucleic acid molecule,
e) contacting the biological sample with a second detectably labeled magnetic particle, wherein the second detectably labeled magnetic particle comprises:
(i) a magnetic core, and
(ii) a coating or lipid membrane comprising a plurality of coupling agent molecules, wherein at least a subset of the coupling agent molecules is coupled with oligonucleotides comprising a second reporter hybridization sequence,
wherein the second reporter hybridization sequence is complementary to the second reporter sequence, whereby the magnetic particle binds to the second reporter sequence via one or more of the oligonucleotide molecules and is thereby associated with the target nucleic acid molecule; and
f) detecting the second detectably labeled magnetic particle at the location in the biological sample.

97. The method of claim 89, wherein oligonucleotide molecules of the detectably labeled magnetic particle hybridize to multiple intermediate probes of the plurality of intermediate probes comprising the reporter sequence.

98. The method of claim 89, wherein the magnetic core comprises a paramagnetic or ferromagnetic material.

99. The method of claim 89, wherein the magnetic core comprises Fe²⁺Fe³⁺₂O₄.

100. The method of claim 89, wherein the magnetic core comprises a magnetite crystal.

101. The method of claim 89, wherein the detectably labeled magnetic particle is a modified magnetosome derived from magnetotactic bacteria.

102. The method of claim 101, wherein the coupling agent is operably linked to a magnetosome membrane protein selected from the group consisting of mamG, mamF, mamD, mamA, and mamC.

103. The method of claim 101, wherein the magnetotactic bacteria are engineered to express the magnetosome membrane protein fused to the capture agent.

104. The method of claim 89, wherein the target nucleic acid molecule is a rolling circle amplification (RCA) product.

105. The method of claim 104, wherein the rolling circle amplification product comprises multiple copies of a barcode sequence, and wherein the plurality of intermediate probes comprises a barcode recognition sequence complementary to the barcode sequence.

106. The method of claim 89, wherein detecting the detectably labeled magnetic particle at a location in the biological sample comprises detecting a single detectably labeled magnetic particle hybridized to the plurality of intermediate probes bound directly to the target nucleic acid molecule.

107. A magnetic particle, comprising:
(i) a magnetic core,
(ii) a detectable label, and
(iii) a plurality of oligonucleotides comprising a reporter hybridization sequence, wherein the reporter hybridization sequence corresponds to the detectable label.

108. A kit, comprising:
a) at least four different species of detectably labeled magnetic particles, wherein each species of detectably labeled magnetic particles comprises: (i) a magnetic core, (ii) a different detectable label, and (iii) oligonucleotides comprising a reporter hybridization sequence corresponding to the detectable label; and
b) a sequential series of intermediate probe panels, wherein each panel comprises multiple intermediate probe species, and wherein each intermediate probe species comprises (i) a barcode recognition sequence complementary to a different barcode sequence, and (ii) a reporter sequence, which can be the same or different from the reporter sequence of a different intermediate probe species in the panel;
wherein the reporter sequence is complementary to the reporter hybridization sequence of one of the species of detectably labeled magnetic particles.
